(19) United States
(12) Patent Application Publication
(10) Pub. No.: US 2006/0115827 A1
(43) Pub. Date: Jun. 1, 2006

(54) Genetic markers for predicting disease and treatment outcome

(75) Inventor: Heinz-Josef Lenz, Altadena, CA (US)

Correspondence Address: FOLEY & LARDNER LLP, 1530 PAGE MILL ROAD, PALO ALTO, CA, 94304, US

(73) Assignee: UNIVERSITY OF SOUTHERN CALIFORNIA, Los Angeles, CA (US)

(21) Appl. No.: 11/173,889

(22) Filed: Jul. 1, 2005

Related U.S. Application Data
(60) Provisional application No. 60/585,019, filed on Jul. 1, 2004.
(60) Provisional application No. 60/653,188, filed on Feb. 14, 2005.
(60) Provisional application No. 60/677,161, filed on May 2, 2005.

(51) Int. Cl. C12Q 1/68; A61K 31/7072; A61K 31/513; A61N 5/00; A61K 39/395

(52) U.S. Cl. 435/6; 514/49; 600/1; 424/155.1; 514/492

(57) ABSTRACT

The invention provides compositions and methods for determining the increased risk for recurrence of certain cancers and the likelihood of successful treatment with one or both of chemotherapy and radiation therapy. The methods comprise determining the type of genomic polymorphism present in a predetermined region of the gene of interest isolated from the subject or patient. Also provided are nucleic acid probes and kits for determining a patient's cancer risk and treatment response.

### CROSS-REFERENCE TO RELATED APPLICATIONS

This application claims the benefit under 35 U.S.C § 119(e) of provisional applications U.S. Ser. Nos. 60/585,019; 60/653,188 and 60/677,161, filed Jul. 1, 2004; Feb. 14, 2005 and May 2, 2005, respectively. The contents of these applications are incorporated by reference into the present disclosure.

### FIELD OF THE INVENTION

This invention relates to the field of pharmacogenomics and specifically to the application of genetic polymorphism to diagnose and treat diseases.

### BACKGROUND OF THE INVENTION

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

Polymorphism also plays a role in determining differences in an individual's response to drugs. Cancer chemotherapy is limited by the predisposition of specific populations to drug toxicity or poor drug response. Thus, for example, pharmacogenetics (the effect of genetic differences on drug response) has been applied in cancer chemotherapy to understand the significant inter-individual variations in responses and toxicities to the administration of anti-cancer drugs, which may be due to genetic alterations in drug metabolizing enzymes or receptor expression. For a review of the use of germline polymorphisms in clinical oncology, see Lenz, H.-J. (2004) J. Clin. Oncol. 22(13):2519-2521.

Polymorphism also has been linked to cancer susceptibility (oncogenes, tumor suppressor genes and genes of enzymes involved in metabolic pathways) of individuals. In patients younger than 35 years, several markers for increased cancer risk have been identified. For example, prostate specific antigen (PSA) is used for the early detection of prostate cancer in asymptomatic younger males. Cytochrome P4501A1 and gluthathione S-transferase M1 genotypes influence the risk of developing prostate cancer in younger patients. Similarly, mutations in the tumor suppressor gene, p53, are associated with brain tumors in young adults.

Results from numerous studies suggest several genes may play a major role in the principal pathways of cancer progression and recurrence, and that the corresponding germ-line polymorphisms may lead to significant differences at transcriptional and/or translational levels.

Moreover, while adjuvant chemotherapy and radiation lead to a noticeable improvement in local control among those with rectal carcinoma, the choice of optimal therapy may be compromised by a wide inter-patient variability of treatment response and host toxicity. Since the rate of inactivation of the administered drug compound may establish its effectiveness in the tumor tissue, genomic variations on different cellular mechanisms that may modify therapy efficacy may influence efficacy. In addition, tumor microenvironment is a critical pathway in cancer progression. Elements of cancer progression controlled by tumor microenvironment genes include angiogenesis, inter-cellular adhesion, mitogenesis, and inflammation. Angiogenesis, which involves the formation of capillaries from preexisting vessels, has been characterized by a complex surge of events involving extensive interchange between cells, soluble factors (e.g. cytokines), and extracellular matrix (ECM) components (Balasubramanian (2002) Br. J. Cancer 87:1057). In addition to its fundamental role in reproduction, development, and wound repair, angiogenesis has been shown to be deregulated in cancer formation (Folkman (2002) Semin. Oncol. 29(6):15).

Improvement in the therapeutic ratio of radiation by targeting tumor cells via a combination of angiogenic blockades and radiotherapy have been implicated in recent studies (Gorski (1999) Cancer Res. 59:3374; Mauceri (1996) Cancer Res. 56:4311; and Mauceri (1998) Nature 394:287).). However, the mechanisms by which tumor cells respond to radiation through these antiangiogenic/vascular agents are yet to be elucidated. Moreover, in light of the fact that oxygen is a potent radiosensitizer, cancer therapy through the combination of ionizing radiation and antiangiogenic/vascular targeting agents may seem counterintuitive since a reduction in tumor vasculature would be expected to decrease tumor blood perfusion and lower oxygen concentration in the tumor (Wachsberger (2003) Clin. Cancer Res. 9:1957).

The interleukin family is known to play an important role in the angiogenic process. Interleukin-8, an inflammatory cytokine with angiogenic potential, has been implicated in cancer progression in a variety of cancer types including colorectal carcinoma, glioblastoma, and melanoma (Yuan (2000) Am. J. Respir. Crit. Care Med. 162:1957). Inter-cellular adhesion plays a major role in both local invasion and metastasis. Cell adhesion molecules (CAMs), which are cell-surface glycoproteins that are crucial for cell-to-cell interactions, have been shown to directly control differentiation, and interruption of normal cell-to-cell contacts has been observed in neoplastic transformation and in metastasis (Edelman (1988) Biochem. 27:3533 and Ruoslahti (1988) Ann. Rev. Biochem. 57:375). Overexpression of ICAM-1 in colorectal cancers has been shown to favor the extravasation and trafficking of cytotoxic lymphocytes toward the neoplastic cells, leading to host defense (Maurer (1998) Int. J. Cancer (Pred. Oncol.) 79:76). A polymorphism in the gene coding for Cox-2 was also studied Cox-2 is involved in prostaglandin synthesis, and stimulates inflammation and mitogenesis; it has been shown to be markedly overexpressed in colorectal adenomas and adenocarcinomas when compared to normal mucosa (Eberhart (1994) Gastro. 107:1183. Another family of genes playing a critical role in angiogenesis is the receptor tyrosine kinase family of fibroblast growth factor receptors. FGFRs are also involved in tumor growth and cell migration. The complex pathways of the tumor microenvironment have become the focus of widespread investigation for their role in tumor progression.

Differences in drug metabolism, transport, signaling and cellular response pathways have been shown to collectively influence diversity in patients' reactions to therapy (Evans (1999) Science 286:487). Metabolism of chemotherapeutic agents and radiation-induced products of oxidative stress, therefore, may play a critical role in treatment response. The GST super-family participates in the detoxification processes of platinum compounds (Ban (1996) Cancer Res. 56:3577 and Goto (1999) Free Rad. Res. 31:549), and was previously associated GSTP1 polymorphism with response to platinum-based chemotherapy (Stoehlmacher (2002) J. Nat. Cancer Inst. 94:936).

Cell cycle regulation provides the foundation for a critical balance between proliferation and cell death, which are important factors in cancer progression. For example, a tumor suppressor gene such as p53 grants the injured cell time to repair its damaged DNA by inducing cell cycle arrest before reinitiating replicative DNA synthesis and/or mitosis (Kastan (1991) Cancer Res. 51:6304). More importantly, when p53 is activated based on DNA damage or other activating factors, it can initiate downstream events leading to apoptosis (Levine (1992) N. Engl. J. Med. 326:1350). The advent of tumor recurrence after radiation therapy depends significantly on how the cell responds to the induced DNA damage; that is, increased p53 function should induce apoptosis in the irradiated cell and thereby prevent proliferation of cancerous cells, whereas decreased p53 function may decrease apoptotic rates.

Finally, DNA repair capacity contributes significantly to the cell's response to chemoradiation treatment (Yanagisawa (1998) Oral Oncol. 34:524). Patient variability in sensitivity to radiotherapy can be attributed to either the amount of damage induced upon radiation exposure or the cell's ability to tolerate and repair the damage (Nunez (1996) Rad. Onc. 39:155). Irradiation can damage DNA directly, or indirectly via reactive oxygen species, and the cell has several pathways to repair DNA damage including double-stranded break repair (DSBR), nucleotide excision repair (NER), and base excision repair (BER). An increased ability to repair direct and indirect damage caused by radiation will inherently lower treatment capability and hence may lead to an increase in tumor recurrence.

### DESCRIPTION OF THE EMBODIMENTS

This invention provides methods to detect polymorphisms that have been determined to be clinically relevant in cancer treatment and prognosis. Clinical relevance includes, but is not specifically limited to patient response to a particular therapy (chemotherapy versus antibody therapy), likelihood of tumor recurrence, survival, sensitivity and toxicity.

In one aspect, the method requires determining the presence or absence of allelic variant of a predetermined gene. In another aspect, it requires determining the identity of a nucleotide of an allelic variant of a predetermined allelic variant. In yet a further embodiment, the method requires determining whether the predetermined gene is over- or under-expressed as compared to a control. In yet a further aspect, one or more of these is identified in the method of this invention.

The genes of interest are selected from those shown to be involved with cancer as described above. For the purpose of illustration only, such genes include, but are not limited to a gene that plays a role in determining differences in an individual's response to a therapy, genes involved with drug metabolism or receptor expression, genes that have been linked to cancer susceptibility (oncogenes, tumor suppressor genes and genes of enzymes involved in metabolic pathways) and genes that are linked to tumor microenvironment such as tumor angiogenesis, inter-cellular adhesion, mitogenesis, and inflammation. Additional genes include, but are not limited to interleukin-8, genes encoding cell adhesion molecules (CAMs) and the Cox-2 gene Another family of genes known to play a role in angiogenesis and therefore cancer is the receptor tyrosine kinase family of fibroblast growth factor receptors.

Yet further examples include, but are not limited to genes involved with metabolism of chemotherapeutic agents and radiation-induced products of oxidative stress, e.g., the GST super-family which participates in the detoxification processes of platinum compounds and associated with response to platinum-based chemotherapy.

Still further examples include genes involved in cell cycle regulation, e.g., a tumor suppressor gene such as p53, and DNA repair capacity.

The invention also provides the tools that can be used to perform the methods of this invention. In one aspect, the tools can include using nucleic acids encompassing the polymorphic region of interest or adjacent to the polymorphic region as probes or primers. In another aspect, the tools are used to detect mRNA levels of a gene of interest. In yet further aspect, antibodies can be used to detect protein expression levels and/or receptor expression levels of the gene of interest.

While the specific experimental embodiments have focused on colorectal carcinoma, the methods of this invention are not so limited. In one aspect, the cancer comprises a cancer or neoplasm that is treatable by use of one or more of platinum-based therapy, fluropyrimidine, CP-11, oxaliplatin, irinotecan, cisplatin, 5-flurouracil (5-FU), radiation and surgical resection. In another aspect, the cancer is treatable by blocking or inhibiting one or more members of the Epidermal Growth Factor Receptor (EGFR) pathway. Non-limiting examples of such cancers include, but are not limited to rectal cancer, colorectal cancer, colon cancer, gastric cancer, lung cancer, and esophageal cancers.

In one aspect, the sample to be tested is the actual tumor tissue. In another aspect the sample can be normal tissue isolated adjacent to the tumor. In a further aspect, the sample is any tissue of the patient, and can include peripheral blood lymphocytes.

In another aspect, the invention comprises administration of an appropriate therapy or combination therapy after identification of the polymorph of interest.

In yet a further embodiment, the invention provides a kit for amplifying and/or for determining the molecular structure of at least a portion of the gene of interest, comprising a probe or primer capable of detecting to the gene of interest and instructions for use. In one embodiment, the probe or primer is capable of detecting to an allelic variant of the gene of interest. In other aspect, the probe or primer is used to determine the expression level of the gene of interest. In yet a further embodiment, the kit contains a molecule, such as an antibody, that can detect the expression product of the gene of interest.

### MODES FOR CARRYING OUT THE INVENTION

The present invention provides methods and kits for determining a subject's cancer risk and likely response to specific cancer treatment by determining the subject's genotype at the gene of interest and/or the level of transcription of a gene of interest. Other aspects of the invention are described below or will be apparent to one of skill in the art in light of the present disclosure.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, cell biology, biochemistry and immunology, which are within the skill of the art. Such techniques are explained fully in the literature for example in the following publications. See, e.g., Sambrook et al. MOLECULAR CLONING: A LABORATORY MANUAL, 2^{nd} edition (1989); CURRENT PROTOCOLS IN MOLECULAR BIOLOGY (F. M. Ausubel et al. eds. (1987)); the series METHODS IN ENZYMOLOGY (Academic Press, Inc., N.Y.); PCR: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1988)); ANIMAL CELL CULTURE (R. I. Freshney ed. (1987)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); IMMUNOCHEMICAL METHODS IN CELL AND MOLECULAR BIOLOGY (Mayer and Walker, eds., Academic Press, London (1987)); HANDBOOK OF EXPERIMENTAL IMMUNOLOGY, Volumes I-IV (D. M. Weir and C. C. Blackwell, eds. (1986)); MANIPULATING THE MOUSE EMBRYO (Cold Spring Harbor Laboratory Press, Cold Spring Harbor, N.Y. (1986)).

### DEFINITIONS

As used herein, certain terms may have the following defined meanings. As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

The term “antigen” is well understood in the art and includes substances which are immunogenic. The EGFR is an example of an antigen. The term as used herein also includes substances which induce immunological unresponsiveness or anergy.

A “native” or “natural” or “wild-type” antigen is a polypeptide, protein or a fragment which contains an epitope and which has been isolated from a natural biological source. It also can specifically bind to an antigen receptor.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention.

The antibodies can be polyclonal or monoclonal and can be isolated from any suitable biological source, e.g., murine, rat, sheep and canine. Additional sources are identified infra.

In one aspect, the “biological activity” means the ability of the antibody to selectively bind its epitope protein or fragment thereof as measured by ELISA or other suitable methods.

The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the VL, VH, CL and CH, domains; a F(ab′)² fragment, a bivalent fragment comprising two Fab fragments linked by a disulfide bridge at the hinge region; a Fd fragment consisting of the VH and CH, domains; a Fv fragment consisting of the VL and VH domains of a single arm of an antibody, a dAb fragment (Ward et al. (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)). Bird et al. (1988) Science 242:423-426 and Huston et al. (1988) Proc. Natl. Acad Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “antibody variant” is intended to include antibodies produced in a species other than a mouse. It also includes antibodies containing post-translational modifications to the linear polypeptide sequence of the antibody or fragment. It further encompasses fully human antibodies.

The term “antibody derivative” is intended to encompass molecules that bind an epitope as defined above and which are modifications or derivatives of a native monoclonal antibody of this invention. Derivatives include, but are not limited to, for example, bispecific, multispecific, heterospecific, trispecific, tetraspecific, multispecific antibodies, diabodies, chimeric, recombinant and humanized.

The term “bispecific molecule” is intended to include any agent, e.g., a protein, peptide, or protein or peptide complex, which has two different binding specificities. The term “multispecific molecule” or “heterospecific molecule” is intended to include any agent, e.g. a protein, peptide, or protein or peptide complex, which has more than two different binding specificities.

The term “heteroantibodies” refers to two or more antibodies, antibody binding fragments (e.g., Fab), derivatives thereof, or antigen binding regions linked together, at least two of which have different specificities.

The term “human antibody” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (V_{L}, V_{H})) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germlne immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

The terms “monoclonal antibody” or “monoclonal antibody composition” as used herein refer to a preparation of antibody molecules of single molecular composition. A monoclonal antibody composition displays a single binding specificity and affinity for a particular epitope.

A “human monoclonal antibody” refers to antibodies displaying a single binding specificity which have variable and constant regions derived from human germline immunoglobulin sequences.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

As used herein, “isotype” refers to the antibody class (e.g., IgM or IgG1) that is encoded by heavy chain constant region genes.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

The terms “protein”, “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “allelic variant of a polymorphic region of the gene of interest” refers to a region of the gene of interest having one of a plurality of nucleotide sequences found in that region of the gene in other individuals.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu, D. Y. et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “genotype” refers to the specific allelic composition of an entire cell or a certain gene, whereas the term “phenotype’ refers to the detectable outward manifestations of a specific genotype.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “mismatches” refers to hybridized nucleic acid duplexes which are not 100% homologous. The lack of total homology may be due to deletions, insertions, inversions, substitutions or frameshift mutations.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and thymidine” are used. It is understood that if the nucleic acid is RNA, a nucleotide having a uracil base is uridine.

The terms “oligonucleotide” or “polynucleotide”, or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

A “polymorphic gene” refers to a gene having at least one polymorphic region.

The term “treating” as used herein is intended to encompass curing as well as ameliorating at least one symptom of the condition or disease. For example, in the case of cancer, treatment includes a reduction in cachexia. Evidence of treatment may be clinical or subclinical.

A “complete response” (CR) to a therapy defines patients with evaluable but non-measurable disease, whose tumor and all evidence of disease had disappeared.

A “partial response” (PR) to a therapy defines patients with anything less than complete response were simply categorized as demonstrating partial response.

“Non-response” (NR) to a therapy defines patients whose tumor or evidence of disease has remained constant or has progressed.

This invention provides a method for selecting a therapeutic regimen or determining if a certain therapeutic regimen is more likely to treat a cancer or is the appropriate chemotherapy for that patient than other available chemotherapies. In general, a therapy is considered to “treat” cancer if it provides one or more of the following treatment outcomes: reduce or delay recurrence of the cancer after the initial therapy; increase median survival time or decrease metastases. The method is particularly suited to determining which patients will be responsive or experience a positive treatment outcome to a chemotherapeutic regimen involving administration of a fluropyrimidine drug such as 5-FU or a platinum drug such as oxaliplatin or cisplatin. Alternatively, the chemotherapy includes administration of a topoisomerase ihibitor such as irinotecan. In a yet further embodiment, the therapy comprises administration of an antibody (as broadly defined herein), ligand or small molecule that binds the Epidermal Growth Factor Receptor (EGFR). These methods are useful to diagnose or predict individual responsiveness to any cancer that has been treatable with these therapies, for example, highly aggressive cancers such as colorectal cancer (CRC).

In one embodiment, the chemotherapeutic regimen further comprises radiation therapy. In an alternate embodiment, the therapy comprises administration of an anti-EGFR antibody or biological equivalent thereof.

The method comprises isolating a suitable cell or tissue sample from the patient and screening for a genomic polymorphism or genotype that has been correlated by the Applicants to be clinically significant. In one aspect, the cancer is a cancer that can be treated by the administration of a chemotherapeutic drug selected from the group consisting of fluoropyrimidine (e.g., 5-FU), oxaliplatin, CPT-11, (e.g., irinotecan) a platinum drug or an anti-EGFR antibody, such as the cetuximab antibody or a combination of such therapies, alone or in combination with surgical resection of the tumor. In another aspect, the cancer is selected from the group consisting of esophageal cancer, gastric cancer, colon cancer, EGFR—positive metastatic colon cancer, rectal cancer, colorectal cancer, lung cancer, and non-small cell lung cancer (NSCLC). In yet a further aspect, the treatment compresses radiation therapy and/or surgical resection of the tumor masses.

In one aspect, the polymorphism is present in a open reading frame (coded) region of the gene, in a “silent” region of the gene, in another it is in the promoter region and in yet another it is in the 3′ untranslated region of the transcript. In yet a further embodiment, the polymorphism increases expression at the mRNA level.

In one embodiment, the tissue is the tumor tissue itself or normal tissue immediately adjacent to the tumor. In yet a further embodiment, any cell expected to carry the gene of interest, when the polymorphism is genetic, such as a peripheral blood lymphocyte isolated from the patient, is a suitable cell or tissue sample.

Genetic polymorphisms that can be predictive of outcome include, but are not limited to polymorphisms occurring in a gene selected from the group consisting of thymidylate synthase gene, VEGF, human glutathione s-transferase P1 gene, epidermal growth factor receptor gene (EGFR), CCND1, ERCC1, Werner locus, TGF-β, XPD, COX-2, Survivin, MnSOD, GPx-1, matrix metalloproteinase gene-1 (MMP-1), Interleukin-8 (IL-8) gene, and Dipyrimidine dehydrogenase (DPD).

This invention also provides a method for determining if a human patient is more likely to experience tumor recurrence after surgical removal of the tumor, by determining the expression level of a gene selected from the group consisting of Dipyrimidine dehydrogenase (DPD), VEGF, Survivin, MnSOD, GPx-1, ERCC1 and EGFR, in a cell or sample isolated from the tumor or cancer cell or tissue or in another embodiment, normal tissue adjacent to the tumor. The expression level is correlated to the expression level within normal levels. In one aspect, overexpression of the gene is predictive to identify patients at risk for tumor recurrence.

Diagnotic Methods

The invention further features predictive medicines, which are based, at least in part, on determination of the identity of the polymorphic region or expression level (or both in combination) of the gene of interest.

For example, information obtained using the diagnostic assays described herein is useful for determining if a subject will respond to cancer treatment of a given type. Based on the prognostic information, a doctor can recommend a regimen (e.g. diet or exercise) or therapeutic protocol, useful for treating cancer in the individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. Expression patterns of individual patients can then be compared to the expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Detection of point mutations can be accomplished by molecular cloning of the specified allele and subsequent sequencing of that allele using techniques known in the art. Alternatively, the gene sequences can be amplified directly from a genomic DNA preparation from the tumor tissue using PCR, and the sequence composition is determined from the amplified product. As described more fully below, numerous methods are available for analyzing a subject's DNA for mutations at a given genetic locus such as the gene of interest.

A detection method is allele specific hybridization using probes overlapping the polymorphic site and having about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244.

In other detection methods, it is necessary to first amplify at least a portion of the gene of interest prior to identifying the allelic variant. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al., (1989) Proc. Natl. Acad. Sci. USA 86:1173-1177), Q-Beta Replicase (Lizardi, P. M. et al. (1988) Bio/Technology 6:1197), or any other nucleic acid amplification method, followed by the detection of the amplified molecules using techniques known to those of skill in the art. These detection schemes are useful for the detection of nucleic acid molecules if such molecules are present in very low numbers.

In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert ((1997) Proc. Natl Acad Sci, USA 74:560) or Sanger (Sanger et al. (1977) Proc. Nat. Acad. Sci, 74:5463). It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number W094/16101, entitled DNA Sequencing by Mass Spectrometry by H. Koster; U.S. Pat. No. 5,547,835 and international patent application Publication Number WO 94/21822 entitled “DNA Sequencing by Mass Spectrometry Via Exonuclease Degradation” by H. Koster; U.S. Pat. No. 5,605,798 and International Patent Application No. PCT/US96/03651 entitled DNA Diagnostics Based on Mass Spectrometry by H. Koster; Cohen et al. (1996) Adv. Chromat. 36:127-162; and Griffin et al. (1993)Appl Biochem Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method Of DNA Sequencing Employing A Mixed DNA-Polymer Chain Probe” and U.S. Pat. No. 5,571,676 entitled “Method For Mismatch-Directed In Vitro DNA Sequencing.”

In some cases, the presence of the specific allele in DNA from a subject can be shown by restriction enzyme analysis. For example, the specific nucleotide polymorphism can result in a nucleotide sequence comprising a restriction site which is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with S1nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

In other embodiments, alterations in electrophoretic mobility is used to identify the particular allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766; Cotton (1993) Mutat. Res. 285:125-144 and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl Acad. Sci USA 86:6230 and Wallace et al. (1979) Nucl. Acids Res. 6:3543). Such allele specific oligonucleotide hybridization techniques may be used for the detection of the nucleotide changes in the polylmorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al. Science 241:1077-1080 (1988). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996)Nucleic Acids Res. 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting the single nucleotide polymorphism in the gene of interest. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. supra, is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al. (1989) Nucl. Acids. Res. 17:7779-7784; Sokolov, B. P. (1990) Nucl. Acids Res. 18:3671; Syvanen, A.-C., et al. (1990) Genomics 8:684-692; Kuppuswamy, M. N. et al. (1991) Proc. Natl. Acad. Sci. (U.S.A.) 88:1143-1147; Prezant, T. R. et al. (1992) Hum. Mutat. 1:159-164; Ugozzoli, L. et al. (1992) GATA 9:107-112; Nyren, P. et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al. (1993) Amer. J. Hum. Genet. 52:46-59).

If the polymorphic region is located in the coding region of the gene of interest, yet other methods than those described above can be used for determining the identity of the allelic variant. For example, identification of the allelic variant, which encodes a mutated signal peptide, can be performed by using an antibody specifically recognizing the mutant protein in, e.g., immunohistochemistry or immunoprecipitation. Antibodies to the wild-type or signal peptide mutated forms of the signal peptide proteins can be prepared according to methods known in the art.

Antibodies directed against wild type or mutant peptides encoded by the allelic variants of the gene of interest may also be used in disease diagnostics and prognostics. Such diagnostic methods, may be used to detect abnormalities in the level of expression of the peptide, or abnormalities in the structure and/or tissue, cellular, or subcellular location of the peptide. Protein from the tissue or cell type to be analyzed may easily be detected or isolated using techniques which are well known to one of skill in the art, including but not limited to Western blot analysis. For a detailed explanation of methods for carrying out Western blot analysis, see Sambrook et al., (1989) supra, at Chapter 18. The protein detection and isolation methods employed herein can also be such as those described in Harlow and Lane, (1988) supra. This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of the peptides or their allelic variants. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the subject polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject has or is at risk of developing disease such as colorectal cancer.

Sample nucleic acid for use in the above-described diagnostic and prognostic methods can be obtained from any cell type or tissue of a subject. For example, a subject's bodily fluid (e.g. blood) can be obtained by known techniques (e.g., venipuncture). Alternatively, nucleic acid tests can be performed on dry samples (e.g., hair or skin). Fetal nucleic acid samples can be obtained from maternal blood as described in International Patent Application No. WO91/07660 to Bianchi. Alternatively, amniocytes or chorionic villi can be obtained for performing prenatal testing.

Diagnostic procedures can also be performed in situ directly upon tissue sections (fixed and/or frozen) of patient tissue obtained from biopsies or resections, such that no nucleic acid purification is necessary. Nucleic acid reagents can be used as probes and/or primers for such in situ procedures (see, for example, Nuovo, G. J. (1992) “PCR In Situ Hybridization: Protocols And Applications”, Raven Press, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

The invention described herein relates to methods and compositions for determining and identifying the allele present at the gene of interest's locus. This information is useful to diagnose and prognose disease progression as well as select the most effective treatment among treatment options. Probes can be used to directly determine the genotype of the sample or can be used simultaneously with or subsequent to amplification. The term “probes” includes naturally occurring or recombinant single- or double-stranded nucleic acids or chemically synthesized nucleic acids. They may be labeled by nick translation, Klenow fill-in reaction, PCR or other methods known in the art. Probes of the present invention, their preparation and/or labeling are described in Sambrook et al. (1989) supra. A probe can be a polynucleotide of any length suitable for selective hybridization to a nucleic acid containing a polymorphic region of the invention. Length of the probe used will depend, in part, on the nature of the assay used and the hybridization conditions employed.

In one embodiment of the invention, probes are labeled with two fluorescent dye molecules to form so-called “molecular beacons” (Tyagi, S. and Kramer, F. R. (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis, L. G. (1998) Science 279:1228-9) as has the use of multiple beacons simultaneously (Marras, S. A. (1999) Genet. Anal. 14:151-6). A quenching molecule is useful with a particular fluorophore if it has sufficient spectral overlap to substantially inhibit fluorescence of the fluorophore when the two are held proixmal to one another, such as in a molecular beacon, or when attached to the ends of an oligonucleotide probe from about 1 to about 25 nucleotides.

Labeled probes also can be used in conjunction with amplification of a polymorphism. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule—quencher molecule pair that specifically anneals to a region of a target polynucleotide containing the poymorphism.

Probes can be affixed to surfaces for use as “gene chips.” Such gene chips can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayyem et al. U.S. Pat. No. 5,952,172 and by Kelley, S. O. et al. (1999) Nucleic Acids Res. 27:4830-4837.

Nucleic Acids

In one aspect, the nucleic acid sequences of the gene's allelic variants, or portions thereof, can be the basis for probes or primers, e.g., in methods for determining the identity of the allelic variant of the polymorphic region. Thus, they can be used in the methods of the invention to determine whether a subject is at risk of developing disease such as colorectal cancer or alternatively, which therapy is most likely to treat an individual's cancer.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers for use in the methods of the invention are nucleic acids which hybridize to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes for use in the methods of the invention are nucleic acids which hybridize to the region of interest and which are not further extended. For example, a probe is a nucleic acid which hybridizes to the polymorphic region of the gene of interest, and which by hybridization or absence of hybridization to the DNA of a subject will be indicative of the identity of the allelic variant of the polymorphic region of the gene of interest.

In one embodiment, primers comprise a nucleotide sequence which comprises a region having a nucleotide sequence which hybridizes under stringent conditions to about: 6, or alternatively 8, or alternatively 10, or alternatively 12, or alternatively 25, or alternatively 30, or alternatively 40, or alternatively 50, or alternatively 75 consecutive nucleotides of the gene of interest.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids which are capable of selectively hybridizing to an allelic variant of a polymorphic region of the gene of interest. Thus, such primers can be specific for the gene of interest sequence, so long as they have a nucleotide sequence which is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules which are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane (see, e.g., Letsinger et al., (1989) Proc. Natl. Acad. Sci. U.S.A. 86:6553-6556; Lemaitre et al., (1987) Proc. Natl. Acad. Sci. 84:648-652; and PCT Publication No. WO 88/0981 0, published Dec. 15, 1988), hybridization-triggered cleavage agents, (see, e.g., Krol et al., (1988) BioTechniques 6:958-976) or intercalating agents (see, e.g., Zon (1988) Pharm. Res. 5:539-549). To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook et al. (1989) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451).

Methods of Treatment

The invention further provides methods of treating subjects having cancer. In one embodiment, the method comprises (a) determining the identity of the allelic variant; and (b) administering to the subject an effective amount of a compound that provides therapeutic benefits for the specific allelic variant.

In one aspect, after determining that antibody therapy alone or in combination with other sutiable therapy is likely to provide a benefit to the patient, the invention further comprises administration of an antibody, fragment, variant or derivative thereof that binds EGFR. The antibodies of this invention are monoclonal antibodies, although in certain aspects, polyclonal antibodies can be utilized. They also can be EGFR-neutralizing functional fragments, antibody derivatives or antibody variants. They can be chimeric, humanized, or totally human. A functional fragment of an antibody includes but is not limited to Fab, Fab′, Fab2, Fab′2, and single chain variable regions. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. So long as the fragment or derivative retains specificity of binding or neutralization ability as the antibodies of this invention it can be used. Antibodies can be tested for specificity of binding by comparing binding to appropriate antigen to binding to irrelevant antigen or antigen mixture under a given set of conditions. If the antibody binds to the appropriate antigen at least 2, 5, 7, and preferably 10 times more than to irrelevant antigen or antigen mixture then it is considered to be specific.

The antibodies also are characterized by their ability to specifically bind to an EGFR epitope. The monoclonal antibodies of the invention can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3×63Ag8.653, Sp2 SA3, Sp2 MAI, Sp2 SS1, Sp2 SA5 U397, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O) or the like, or heteromyelomas, fusion products thereof, or any cell or fusion cell derived therefrom, or any other suitable cell line as known in the art (see, e.g., www.atcc.org, www.lifetech.com., and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing-heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1977) Microbiol. Immunol. 41:901-907 (1997); Sandhu et al., (1996) Crit. Rev. Biotechnol. 16:95-118; Eren et al. (1998) Immunol. 93:154-161 that are capable of producing a repertoire of human antibodies, as known in the art and/or as described herein. Such techniques, include, but are not limited to, ribosome display (Hanes et al. (1997) Proc. Natl. Acad. Sci. USA, 94:4937-4942; Hanes et al., (1998) Proc. Natl. Acad. Sci. USA, 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al., Proc. Natl. Acad. Sci. USA (1996) 93:7843-7848); gel microdroplet and flow cytometry (Powell et al. (1990) Biotechnol. 8:333-337; One Cell Systems, (Cambridge, Mass).; Gray et al. (1995) J. Imm. Meth. 182:155-163; Kenny et al. (1995) Bio/Technol. 13:787-790); B-cell selection (Steenbakkers et al. (1994) Molec. Biol. Reports 19:125-134 (1994).

Antibody variants of the present invention can also be prepared using delivering a polynucleotide encoding an antibody of this invention to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody variant” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

Antibody variants also can be prepared by delivering a polynucleotide of this invention to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al., Adv. Exp. Med. Biol. (1999) 464:127-147 and references cited therein. Antibody variants have also been produced in large amounts from transgenic plant seeds including antibody fragments, such as single chain antibodies (scFv's), including tobacco seeds and potato tubers. See, e.g., Conrad et al.(1998) Plant Mol. Biol. 38:101-109 and reference cited therein. Thus, antibodies of the present invention can also be produced using transgenic plants, according to know methods.

Antibody derivatives can be produced, for example, by adding exogenous sequences to modify immunogenicity or reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

In general, the CDR residues are directly and most substantially involved in influencing antigen binding. Humanization or engineering of antibodies of the present invention can be performed using any known method, such as but not limited to those described in U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel, N. D. et al. (2000) Infection and Immunity April 2000:1820-1826; Gallo, M. L. et al. (2000) European J. of Immun. 30:534-540; Green, L. L. (1999) J. of Immun. Methods 231:11-23; Yang, X-D et al. (1999A) J. of Leukocyte Biology 66:401-410; Yang, X-D (1999B) Cancer Research 59(6):1236-1243; Jakobovits, A. (1998) Advanced Drug Delivery Reviews 31:3342; Green, L. and Jakobovits, A. (1998) J. Exp. Med. 188(3):483-495; Jakobovits, A. (1998) Exp. Opin. Invest. Drugs 7(4):607-614; Tsuda, H. et al. (1997) Genomics 42:413421; Sherman-Gold, R.-(1997). Genetic Engineering News 17(14); Mendez, M. et al. (1997) Nature Genetics 15:146-156; Jakobovits, A. (1996) Weir's Handbook of Experimental Immunology, The Integrated Immune System Vol. IV, 194.1-194.7; Jakobovits, A. (1995) Current Opinion in Biotechnology 6:561-566; Mendez, M. et al. (1995) Genomics 26:294-307; Jakobovits, A. (1994) Current Biology 4(8):761-763; Arbones, M. et al. (1994) Immunity 1(4):247-260; Jakobovits, A. (1993) Nature 362(6417):255-258; Jakobovits, A. et al. (1993) Proc. Natl. Acad. Sci. USA 90(6):2551-2555; Kucherlapati, et al. U.S. Pat. No. 6,075,181.)

Human monoclonal antibodies can also be produced by a hybridoma which includes a B cell obtained from a transgenic nonhuman animal, e.g., a transgenic mouse, having a genome comprising a human heavy chain transgene and a light chain transgene fused to an immortalized cell.

The antibodies of this invention also can be modified to create chimeric antibodies. Chimeric antibodies are those in which the various domains of the antibodies' heavy and light chains are coded for by DNA from more than one species. See, e.g., U.S. Pat. No.: 4,816,567.

The term “antibody derivative” also includes “diabodies” which are small antibody fragments with two antigen-binding sites, wherein fragments comprise a heavy chain variable domain (V) connected to a light chain variable domain (V) in the same polypeptide chain (VH V). (See for example, EP 404,097; WO 93/11161; and Hollinger et al., (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen.)

The term “antibody derivative” further includes “linear antibodies”. The procedure for making the is known in the art and described in Zapata et al. (1995) Protein Eng. 8(10):1057-1062. Briefly, these antibodies comprise a pair of tandem Fd segments (V—C 1-VH —C1) which form a pair of antigen binding regions. Linear antibodies can be bispecific or monospecific.

The antibodies of this invention can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

[Antibodies of the present invention include naturally purified products, products of chemical synthetic procedures, and products produced by recombinant techniques from a eukaryotic host, including, for example, yeast, higher plant, insect and mammalian cells, or alternatively from a prokaryotic cells as described above.

Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; Iy207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

Antibodies can also be used in immunohistochemical assays to detect the presence or expression level of a protein of interest. They are further useful to detect the presence or absence of EGFR in a patient sample. In these and other aspects of this invention, it will be useful to detectably or therapeutically label the antibody. Methods for conjugating antibodies to these agents are known in the art. For the purpose of illustration only, antibodies can be labeled with a detectable moiety such as a radioactive atom, a chromophore, a fluorophore, or the like. With respect to preparations containing antibodies covalently linked to organic molecules, they can be prepared using suitable methods, such as by reaction with one or more modifying agents. Examples of such include modifying and activating groups. A “modifying agent” as the term is used herein, refers to a suitable organic group (e.g., hydrophilic polymer, a fatty acid, a fatty acid ester) that comprises an activating group. Specific examples of these are provided supra. An “activating group” is a chemical moiety or functional group that can, under appropriate conditions, react with a second chemical group thereby forming a covalent bond between the modifying agent and the second chemical group. Examples of such are electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., BIOCONJUGATE TECHNIQUES, Academic Press: San Diego, Calif. (1996)). An activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid.

The modified antibodies of the invention can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis. See generally, Hermanson, G. T., BIOCONJUGATE TECHNIQUES, Academic Press: San Diego, Calif. (1996).

Kits

As set forth herein, the invention provides diagnostic methods for determining the type of allelic variant of a polymorphic region present in the gene of interest or the expression level of a gene of interest. In some embodiments, the methods use probes or primers comprising nucleotide sequences which are complementary to the polymorphic region of the gene of interest. Accordingly, the invention provides kits for performing these methods.

In an embodiment, the invention provides a kit for determining whether a subject responds to cancer treatment or alternatively one of various treatment options. The kits contain one of more of the compositions described above and instructions for use. As an example only, the invention also provides kits for determining response to cancer treatment containing a first and a second oligonucleotide specific for the polymorphic region of the gene. Oligonucleotides “specific for” a genetic locus bind either to the polymorphic region of the locus or bind adjacent to the polymorphic region of the locus. For oligonucleotides that are to be used as primers for amplification, primers are adjacent if they are sufficiently close to be used to produce a polynucleotide comprising the polymorphic region. In one embodiment, oligonucleotides are adjacent if they bind within about 1-2 kb, and preferably less than 1 kb from the polymorphism. Specific oligonucleotides are capable of hybridizing to a sequence, and under suitable conditions will not bind to a sequence differing by a single nucleotide.

The kit can comprise at least one probe or primer which is capable of specifically hybridizing to the polymorphic region of the gene of interest and instructions for use. The kits preferably comprise at least one of the above described nucleic acids. Preferred kits for amplifying at least a portion of the gene of interest comprise two primers, at least one of which is capable of hybridizing to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard, T. (1986) “An Introduction to Radioimmunoassay and Related Techniques” Elsevier Science Publishers, Amsterdam, The Netherlands; Bullock, G. R. et al., “Techniques in Immunocytochemistry” Academic Press, Orlando, Fla. Vol. 1 (1982), Vol. 2 (1983), Vol. 3 (1985); Tijssen, P., (1985) “Practice and Theory of Immunoassays: Laboratory Techniques in Biochemistry and Molecular Biology”, Elsevier Science Publishers, Amsterdam, The Netherlands.

The test samples used in the diagnostic kits include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region of the gene of interest.

As amenable, these suggested kit components may be packaged in a manner customary for use by those of skill in the art. For example, these suggested kit components may be provided in solution or as a liquid dispersion or the like.

Other Uses for the Nucleic Acids of the Invention

The identification of the allele of the gene of interest can also be useful for identifying an individual among other individuals from the same species. For example, DNA sequences can be used as a fingerprint for detection of different individuals within the same species (Thompson, J. S. and Thompson, eds., (1991) “Genetics in Medicine”, W B Saunders Co., Philadelphia, Pa.). This is useful, e.g., in forensic studies.

The invention now being generally described, it will be more readily understood by reference to the following examples which are included merely for purposes of illustration of certain aspects and embodiments of the present invention, and are not intended to limit the invention.

### EXPERIMENTAL EXAMPLES

### Example 1

### Association of Polymorphism and Clinical Outcome of EGFR-Positive Cancer Patients Treated with Epidermal Growth Factor Receptor (EGFR) inhibitor, Cetuximab(C225)

This study identifies genomic polymorphisms of the EGFR pathway are useful as molecular markers to predict response to EGFR inhibitors, overall survival and toxicity. This study demonstrates that certain gene polymorphisms involved in the EGFR pathway, CyclinD1 (CCND1) A870G and EGF A61G, are associated with overall survival in metastatic CRC patients treated with the EGFR inhibitor cetuximab. When the analysis of Cyclin DI and EGF polymorphisms were combined together, patients with two favorable genotypes (EGF any A allele and CCND1 any G allele) showed a median survival time of 12 month (95% C.l 4.8-15.2), while patients with any unfavorable genotypes (EGF GG or CylinDI AA) survived only 4.4 months (95% C.l 2.1-5.7).(p=0.004, logrank test).

Patients

Thirty-nine patients with histopathologically confirmed metastatic CRC, who either failed at least two prior chemotherapy regimens or failed adjuvant therapy plus one chemotherapy regimen for metastatic disease (provided the patient progressed within 6 months of completing adjuvant therapy), were included in this study. Patients were enrolled from October of 2002 to March of 2003 at the University of Southern Californial/Norris Comprehensive Cancer Center (USC/NCCC) of Los Angeles. These 39 patients were drawn from the Phase II open-label multi-center study (IMCL-0144) of Cetuximab (C225), which included a total of 346 patients. This study was conducted at USC/Norris Comprehensive Cancer Center and was approved by the Institutional Review Board of the University of Southern California for Medical Sciences. All patients showed immunhistochemical evidence of EGFR expression in their tumor samples.

Patients were infused with cetuximab at standard loading dose 400 mg/m² over a two-hour period, followed by weekly infusion of 250 mg/m² treatment over one-hour period. Treatment was continued until progression of disease or toxicity occurred, and patients were evaluated every six weeks for tumor response.

All patients signed an informed consent for tissue and blood collection for the study of molecular correlates. Blood samples were collected before chemotherapy begin.

A peripheral blood sample was collected from each patient, and genomic DNA was extracted from white blood cells using the QiaAmp kit (Qiagen, Valencia, Calif.). The Cox-2 G765C polymorphism, the CyclinD1A870G polymorphism, the HER1 G497A polymorphism, the IL-8 T251A polymorphism, the EGF 5′UTR A61G polymorphism, and the VEGF C936T polymorphisms were all tested by the PCR-RFLP method. Briefly, forward and reverse primers were used for PCR amplification, PCR product was digested by restriction enzyme (New England Biolabs, Maryland, USA), and alleles were separated on 4% Nusieve ethidium bromide-stained agrose gel. Forward and reverse primers, restriction enzymes, and annealing temperatures for specific gene polymorphisms are listed in Tables 1A and 1B.

TABLE 1A
Primers

| | Forward primer | Reverse Primer |
|---|---|---|
| Gene | (5′-3′) | (5′-3′) |
| CCND1 | GTGAAGTTCATTTCCAATCC | GGGACATCACCCTCACTTAC |
| | GC | |
| COX-2 | CCGCTTCCTTTGTCCATCAG | GGCTGTATATCTGCTCTATA |
| | | TGC |
| EGFR 497 | TGCTGTGACCCACTCTGTCT | CCAGAAGGTTGCACTTGTCC |
| EGF | TGTCACTAAAGGAAAGGA | TTCACAGAGTTTAACAGCCC |
| IL-8 | TTGTTCTAACACCTGCCACT | GGCAAACCTGAGTCATCACA |
| | CT | |
| VEGF | ACACCATCACCATCGACAGA | TCGGTGATTTAGCAGCAAGA |

TABLE 1B
Annealing temperature and restriction enzymes

| | Annealing | |
|---|---|---|
| | Temperature | Restriction |
| Gene | (° C.) | enzymes |
| CCND1 | 55 | ScrF I |
| COX-2 | 55 | AciI |
| EGFR 497 | 59 | BstNI |
| EGF | 51 | AluI |
| IL-8 | 60 | MfeI |
| VEGF | 60 | NIaIII |

Abbreviations:
Cox-2, cyclooxygenase 2;
CCND1, cyclin D1;
EGFR, epidermal growth factor receptor;
IL-8, interleukin 8;
VEGF, vascular endothelial growth factor

As an example, the EGFR (CA)ₙ repeat polymorphism was tested by a 5′-end [γ-³³P]ATP-labeled PCR protocol. Briefly, 100 ng gDNA, 200 μM dNTP's, 1.0 μM 5′ ³³p-γATP end-labeled reverse primer, 1.0 μM unlabeled forward primer, 0.75 U Taq polymerase (Perkin Elmer) and PCR buffer (10 mM Tris-HC1 pH 8.3, 50 mM KC1, 1.5 mM MgCl₂) were used together in a final PCR volume of 15 μL. The reaction was carried out for 28 cycles with denaturation at 94° C. (1 min), annealing at 55° C. (1 min), and extension at 72° C. (2 min). The reaction products were separated on a 6% denaturing polyacrylamide DNA sequencing gel, which was vacuum blotted for one hour at 80° C. and exposed to an XAR film (Eastman-Kodak Co., Rochester, N.Y.) overnight. The exact number of the CA repeats was confirmed by direct sequencing.

Paraffin embedded tumor blocks were used for immunhistochemistry. EGFR immunnoreactivity was investigated at a central laboratory using the EGFR pharmDX™ (DakoCytomation, Glostrup, Denmark). The intensity of membranous immunostaining was defined as: weak (score 1+), moderate (score 2+), or strong (score 3+).

Statistical Analysis

Objective tumor response, toxicity (acne-like rash), and overall survival under Cetuximab chemotherapy treatment were the primary endpoints. The overall survival time was calculated as the period from the first day of Cetuximab infusion until death from any cause, or until the date of the last follow-up, at which point data were censored.

The associations of each polymorphism with tumor response and toxicity were examined using contingency tables and the Fisher's exact test. The association of each polymorphism with survival was analyzed individually using Kaplan-Meier plots and the log-rank test. In the univariate analysis, the relative risk (RR) ratio and its associated 95 percent confidence interval (95% Cl) were based on the log-rank test.

All reported P values were two-sided. All analyses were performed using the SAS statistical package version 8.2 [SAS Institute Inc. S. SAS/STAT® User's Guide, Version 8. Cary, N.C.: SAS Institute Inc., 1999] and Epilog Plus Version 1.0 [Epicenter Software E. Epilog windows user guide and procedures. Pasadena, Calif.: Epicenter Software, 1999].

Results

Twenty-one (21) women and 18 men with a median age of 64 years (range 35-83) were enrolled in this study. Thirty-one (79%) of these patients were Caucasian, six (15%) were Asian, and two (5%) were Hispanic. All (100%) patients were assessable for association between EGFR expression (detected by immunhistochemical staining) and clinical outcome. At the time of analysis, three patients were still alive, the range for those 3 patients were 2.5-10.6 months. The median survival time was 5.5 months (95% Cl, 2.7 to 8.7 months). Under cetuximab treatment, two (6%) patients showed partial response (PR), 21 (60%) patients had stable disease (SD), and 12 (34%) patients had progressive disease (PD), while no patient showed complete response. For four patients the response was inevaluable. Skin reactions were observed in 85% of the 39 patients, where 12 patients (31%) had a grade 1 reactions, 20(51%) patients had grade 2 reactions, and one patient (3%) had a grade 3 reaction (see Table 2).

TABLE 2
Baseline information for patients treated with Cetuximab in a
protocol (3C-02-3) (N = 39)

| Characteristics | Frequency | % |
|---|---|---|
| Median age, years (range) | 64 | 35-83 |
| Gender | | |
| Female | 21 | 54% |
| Male | 18 | 46% |
| Race | | |
| Asian | 6 | 15% |
| Caucasian | 31 | 79% |
| Hispanic | 2 | 5% |
| Anatomical Site | | |
| Colon | 29 | 74% |
| Rectosigmoid | 5 | 13% |
| Rectum | 5 | 13% |
| Histology | | |
| Adenocarcinoma | 31 | 79% |
| Mucinous adenocarcinoma | 7 | 18% |
| Signet cell carcinoma | 1 | 3% |
| Response | | |
| Partial response | 2 | 6% |
| Stable disease | 21 | 60% |
| Progressive disease | 12 | 34% |
| Inevaluable/early off study | 4 | |
| Toxicity (rash) | | |
| Grade 0 | 6 | 15% |
| Grade 1 | 12 | 31% |
| Grade 2 | 20 | 51% |
| Grade 3 | 1 | 3% |

There was no significant correlation between skin toxicity and response. However, patients with a grade 2-3 skin reaction had a significantly longer median progression free survival of 3.2 months (95% Cl, 2.4 to 4.6 months), as compared with the median 1.3 months of patients that had a grade 0-1 toxicity (95% Cl, 1.1 to 2.0 months; P=0.001, log-rank test). Patients with skin reaction toxicity of grade 2-3 also experienced a significantly longer survival (median 7.7 months, 95% Cl, 4.4 to 15.0 months) than patients with grade 1-2 toxicity (median 2.2 months, 95% Cl, 1.8 to 5.7 months)(P=0.049, log-rank test).

Twenty-three patients (59%) had a weak EGFR staining (1+intensity), 11 (28%) patients had a moderate (2+intensity), and 5 (13%) patients had a strong staining (3+intensity). There was no association of EGFR staining with response, survival or toxicity.

The CyclinDI A870G polymorphism showed significant association with overall survival. Patients with the M homozygous genotype survived a median time of 2.3 months (95% C.l 2.1, 5.7), compared to the median 7.8 months survived by those with any G allele (AG, GG genotype)(95% C.l 4.4, 13.5), (p=0.019, logrank test). The EGF A61 G polymorphism also showed a trend of association with overall survival. Patients with the homozygous M genotype survived a median time of 15.0 months (95% C.l 2.3, 15.9), the homozygous GG genotype 2.3 months (95% C.l 1.8, 7.3), and the heterozygous AG genotype 5.7 months (95% C.l 2.7, 10.7) (p=0.07, logrank test). In a combination analysis of CyclinDI and EGF polymorphisms, patients with two favorable genotypes (EGF any A allele and CCNDI any G allele) were found to have a median survival time of 12 month (95% C.l 4.8-15.2), whereas patients with any unfavorable genotypes (EGF G(S or CyclinDI M) survived only 4.4 months (95% C.l 2.1-5.7).(p=0.004, lograa test). Other polymorphisms did not show statistic significant association with overall survival. (See Table 4)

TABLE 3
Genomic polymorphisms and clinical outcome among patients in protocol 3C-02-3

| | | | | | | | Progression-Free | | | |
|---|---|---|---|---|---|---|---|---|---|---|
| | | | | | | | survival | | Overall Survival^{†} | |
| | | | | | Toxicity | | Median, | Relative | Median, | Relative |
| | Response | | | | Grade | Grade | Mo | Risk | Mo | Risk |
| | N | PR | SD | PD | 0-1 | 2-3 | (95% CI) | (95% CI) | (95% CI) | (95% CI) |
| Overall | 39 | 2 (6%) | 21 (60%) | 12 (34%) | 18 (46%) | 21 (54%) | 2.4 (1.4, 3.7) | | 5.5 (2.7, 8.7) | |
| EGF | | | | | | | | | | |
| A/A | 13 | 2 (18%) | 6 (55%) | 3 (27%) | 5 (38%) | 8 (62%) | 2.4 (1.4, 5.0) | 1 (Reference) | 15.0 (2.3, 15.9) | 1 (Reference) |
| A/G | 17 | 0 (0%) | 11 (65%) | 6 (35%) | 8 (47%) | 9 (53%) | 2.4 (1.1, 4.0) | 1.27 (0.60-2.66) | 5.7 (2.7, 10.7) | 1.66 (0.75-3.68) |
| G/G | 4 | 0 (0%) | 2 (50%) | 2 (50%) | 2 (50%) | 2 (50%) | 1.3 (1.1, 2.4) | 2.24 (0.67-7.53) | 2.3 (1.8, 7.3) | 3.30 (0.92-11.9) |
| Missing | 5 | | | | | | | | | |
| P value* | | 0.49 | | | 0.89 | | | 0.32 | | 0.070 |
| Cox-2 G-765C | | | | | | | | | | |
| G/G | 20 | 1 (5%) | 11 (58%) | 7 (37%) | 11 (55%) | 9 (45%) | 2.4 (1.3, 3.7) | 1 (Reference) | 4.8 (2.3, 8.7) | 1 (Reference) |
| G/C | 12 | 1 (8%) | 7 (58%) | 4 (33%) | 3 (25%) | 9 (75%) | 2.4 (1.1, 4.4) | 0.88 (0.44-1.76) | 10.7 (4.5, 15.2) | 0.66 (0.31-1.39) |
| C/C | 2 | 0 (0%) | 1 (50%) | 1 (50%) | 0 (0%) | 2 (100%) | | | | |
| Missing | 5 | | | | | | | | | |
| P value* | | 1.00 | | | 0.15 | | | 0.70 | | 0.24 |
| EGFR 497 | | | | | | | | | | |
| A/A, A/G | 15 | 2 (13%) | 7 (47%) | 6 (40%) | 5 (40%) | 9 (60%) | 2.4 (1.1, 3.7) | 1 (Reference) | 5.5 (2.3, 12.0) | 1 (Reference) |
| G/G | 20 | 0 (0%) | 12 (67%) | 6 (33%) | 9 (45%) | 11 (55%) | 2.3 (1.4, 3.7) | 0.95 (0.48-1.88) | 5.7 (2.7, 15.2) | 0.65 (0.30-1.41) |
| Missing | 4 | | | | | | | | | |
| P value* | | 0.29 | | | 1.00 | | | 0.88 | | 0.20 |
| EGFR (CA)ₙ | | | | | | | | | | |
| repeat | | | | | | | | | | |
| Both | 16 | 2 (13%) | 7 (47%) | 6 (40%) | 5 (31%) | 11 (69%) | 2.0 (1.3, 4.6) | 1 (Reference) | 4.8 (2.2, 15.0) | 1 (Reference) |
| repeats < 20 | | | | | | | | | | |
| Any | 18 | 0 (0%) | 11 (65%) | 6 (35%) | 10 (56%) | 8 (44%) | 2.4 (1.1, 3.7) | 1.51 (0.73-3.12) | 5.7 (4.4, 8.7) | 1.21 (0.59-2.50) |
| repeats ≧ 20 | | | | | | | | | | |
| Missing | 5 | | | | | | | | | |
| P value | | 0.29 | | | 0.19 | | | 0.20 | | 0.58 |
| Cyclin D1 | | | | | | | | | | |
| Any | 27 | 2 (8%) | 16 (62%) | 8 (31%) | 10 (37%) | 17 (63%) | 2.4 (1.4, 3.7) | 1 (Reference) | 8.7 (4.4, 13.5) | 1 (Reference) |
| G(AG, GG) | | | | | | | | | | |
| A/A | 8 | 0 (0%) | 3 (43%) | 4 (57%) | 5 (63%) | 3 (38%) | 1.1 (1.0, 4.4) | 1.43 (0.64-3.20) | 2.3 (2.1, 5.7) | 2.51 (0.94-6.66) |
| Missing | 4 | | | | | | | | | |
| P value | | 0.61 | | | 0.51 | | | 0.54 | | 0.019 |
| IL-8 | | | | | | | | | | |
| AA | 8 | 0 (0%) | 4 (50%) | 4 (50%) | 3 (38%) | 5 (63%) | 1.3 (1.0, 4.4) | 1 (Reference) | 5.7 (2.1, 15.2) | 1 (Reference) |
| AT | 13 | 0 (0%) | 10 (77%) | 3 (23%) | 6 (46%) | 7 (54%) | 2.4 (2.0, 4.6) | 0.61 (0.24-1.55) | 8.7 (4.4, 15.9) | 0.98 (0.37-2.62) |
| TT | 14 | 2 (17%) | 5 (42%) | 5 (42%) | 6 (43%) | 8 (57%) | 2.0 (1.1, 3.7) | 0.98 (0.40-2.36) | 3.4 (2.3, 12.0) | 1.70 (0.64-4.51) |
| Missing | 4 | | | | | | | | | |
| P value | | 0.25 | | | 1.00 | | | 0.35 | | 0.28 |
| VEGF-936 | | | | | | | | | | |
| CC | 30 | 2 (7%) | 16 (55%) | 11 (38%) | 11 (37%) | 19 (63%) | 2.4 (1.3, 3.7) | 1 (Reference) | 7.3 (4.4, 12.1) | 1 (Reference) |
| CT | 5 | 0 (0%) | 3 (75%) | 1 (25%) | 4 (80%) | 1 (20%) | 2.0 (1.1, 10) | 0.73 (0.26-2.05) | 2.3 (2.0, 17.9) | 1.17 (0.44-3.10) |
| Missing | 4 | | | | | | | | | |
| P value | | 1.00 | | | 0.14 | | | 0.47 | | 0.74 |
| Staining | | | | | | | | | | |
| 1+ | 23 | 1 (5%) | 14 (67%) | 6 (29%) | 12 (52%) | 11 (48%) | 2.4 (1.8, 3.7) | 1 (Reference) | 5.7 (2.3, 10.7) | 1 (Reference) |
| 2-3+ | 16 | 1 (7%) | 7 (50%) | 6 (43%) | 6 (38%) | 10 (62%) | 1.4 (1.1, 3.7) | 1.11 (0.58-2.12) | 4.8 (2.3, 12.0) | 1.35 (0.65-2.80) |
| P value | | 0.64 | | | 0.52 | | | 0.74 | | 0.36 |
| Toxicity | | | | | | | | | | |
| Grade 0-1 | 18 | 0 (0%) | 7 (50%) | 7 (50%) | | | 1.3 (1.1, 2.0) | 1 (Reference) | 2.2 (1.8, 5.7) | 1 (Reference) |
| Grade 2-3 | 21 | 2 (10%) | 14 (67%) | 5 (24%) | | | 3.2 (2.4, 4.6) | 0.40 (0.19-0.85) | 7.7 (4.4, 15.0) | 0.53 (0.27-1.04) |
| P value | | 0.28 | | | | | | 0.001 | | 0.049 |

*P values were based on Fisher's exact tests and Log-rank tests for response and time-to-event variables, respectively.
^{†}Three patients were still alive at the time of analysis: the range of follow-up for those 3: 2.5-10.6 months.

Gene Polymorphism and Response to Cetuximab, Skin Toxicity

No association between gene polymorphism and patients response to cetuximab was found for COX-2 G-765C (p=0.15) and VEGF C936T. However these polymorphisms (p=0.14) show trend of associations with skin toxicity. (See Table 3)

TABLE 4
Combined analysis of association between EGF and Cyclin D1
polymorphisms and clinical outcome.

| | | Any one | |
|---|---|---|---|
| | Two favorable | unfavorable | |
| | polymorphisms^{a} | polymorphism | P |
| Clinical Outcome | (n = 23) | (n = 11) | value |
| Response | | | |
| Partial Response | 2 (9%) | 0 | |
| Stable Disease | 14 (64%) | 5 (50%) | 0.39 |
| Progressive Disease | 6 (27%) | 5 (50%) | |
| Toxicity | | | |
| Grade 0-1 | 9 (39%) | 6 (55%) | 0.52 |
| Grade 2-3 | 14 (61%) | 5 (45%) | |
| Progression-Free survival | | | |
| Median survival (95% CI) | 3.7 (2.0-3.7) | 2.0 (1.1-10.1) | |
| Relative Risk (95% CI) | 1 (Reference) | 1.63 (0.77-3.44) | 0.16 |
| Overall survival | | | |
| Median survival | 12.0 (4.8-15.2) | 4.4 (2.1-5.7) | |
| (95% CI), MO | | | |
| Relative Risk (95% CI) | 1 (Reference) | 2.65 (1.05-6.68) | 0.004 |

^{a}Favorable genotypes of EGF (A/A, A/G), Cyclin D1 (G/G, A/G)

### Example 2

### Multi-Factorial Analysis of Metastatic Colorectal Cancer Patients Treated with Cetuximab

This study investigated whether mRNA expression levels of members of the EGFR signaling pathway, e.g., Cyclin D1 (CCNDI), cyclooxygenase 2 (COX-2), epidermal growth factor receptor (EGFR), Interleukin 8 (IL-8) and vascular endothelial growth factor (VEGF), are associated with the clinical outcome in patients with EGFR-expressing metastatic colorectal cancer (CRC) treated with cetuximab.

Patients

The same patient sample of Experimental Example 2 was used for this study.

Sample Preparation

For the evaluation of gene expression levels, tumor samples were obtained from the primary colorectal tumor or from metastatic site of the liver at the time of diagnosis. Paraffin-embedded tumor blocks were reviewed for quality and tumor content by a pathologist. Ten (10) micrometer thick sections were obtained from the identified areas with the highest tumor concentration. Sections were mounted on uncoated glass slides. For histology diagnosis, three representative sections, consisting of the beginning, the middle and the end of sections of the tissue were stained with H&E by the standard method. Before microdissection, sections were deparafinized in xylene for 10 minutes and hydrated with 100%, 95% and finally 70% ethanol. Then they were washed in H₂O for 30 seconds. Afterwards, they were stained with nuclear fast red (NFR, American MasterTech Scientific, Inc., Lodi, Calif.) for 20 seconds and rinsed in H₂O for 30 seconds. Samples were then dehydrated with 70% ethanol, 95% ethanol and 100% ethanol for 30 seconds each, followed by xylene for 10 minutes. The slides were then completely air-dried. If the histology of the samples was homogeneous and contained more than 90% tissue of interest, samples were dissected from the slides using a scalpel. All other sections of interest were selectively isolated by laser capture microdissection (P.A.L.M. Microsystem, Leica, Wetzlar, Germany) according to the standard procedure. The dissected particles of tissue were transferred to a reaction tube containing 400 μl of RNA lysis buffer.

RNA isolation from paraffin-embedded samples was done according to a proprietary procedure of Response Genetics, Inc. (Los Angeles, Calif.; U.S. Pat. No. 6,248,535). cDNA was prepared as descried in Lord, R. V. et al. (2000) J. Gastrointest. Surg. 4:135-142.

Quantitation of COX-2, CCND1, EGFR, IL8, VEGF and an internal reference gene (β-actin) was done using a fluorescence based real-time detection method (ABI PRISM 7900 Sequence detection System (TagMan®) Perkin-Elmer (PE) Applied Biosystem, Foster City, Calif., USA). The PCR reaction mixture consisted 1200 nM of each primer, 200 nM probe, 0.4 U of AmpliTaq Gold Polymerase, 200 nM each dATP, dCTP, dGTP, dTTP, 3.5 mM MgCl₂ and 1×Taqman Buffer A containing a reference dye, to a final volume of 20 μl (all reagents from PE Applied Biosystems, Foster City, Calif., USA). Cycling conditions were 50° C. for 2 min, 95° C. for 10 min, followed by 46 cycles at 95° C. for 15s and 60° C. for 1 min. The primers and probes used are listed in Table 5. (SEQ ID NOS. ______ to ______).

TABLE 5
Primers and Probes sequences

| | Gen Bank | Forward primer | Reverse Primer | Taqman probe |
|---|---|---|---|---|
| Gene | Accession | (5′-3′) | (5′-3′) | (5′-3′) |
| β-actin | NM_001101 | GAGCGCGGCTACAGCTT | TCCTTAATGTCACGCACGATTT | ACCACCACGGCCGAGCGG |
| Cox-2 | NM_000963 | GCTCAAACATGATGTTTGCATTC | GCTGGCCCTCGCTTATGA | TGCCCAGCACTTCACGCATCAGTT |
| CCND1 | NM_053056 | TGCATGTTCGTGGCCTCTAA | TCGGTGTAGATGCACAGCTTCT | AAGGAGACCATCCCCCTGACGGC |
| EGFR | NM_005228 | TGCGTCTCTTGCCGGAAT | GGCTCACCCTCCAGAAGCTT | ACGCATTCCCTGCCTCGGCTG |
| IL-8 | NM_000584 | CAGCTCTGTGTGAAGGTGCAGTT | GGGTGGAAAGGTTTGGAGTATGTC | TGCACTGACATCTAAGTTCTTTAGCACTCCTT |
| | | | GGC | |
| VEGF | NM_003376 | AGTGGTCCCAGGCTGCAC | TCCATGAACTTCACCACTTCGT | ATGGCAGAAGGAGGAGGGCAGAATCA |

Abbreviations: Cox-2, cyclooxygenase 2; CCND1, cyclin D1; EGFR, epidermal growth factor receptor; IL-8, interleukin 8; VEGF, vascular endothelial growth factor

Table 2 supra, provides Demographic and clinical parameters of patients with metastatic CRC treated with cetuximab.

TagMan® measurements yield Ct values that are inversely proportional to the amount of cDNA in the tube, i.e., a higher Ct value means it requires more PCR cycles to reach a certain level of detection. Gene expression values (relative mRNA levels) are expressed as ratios (differences between the Ct values) between the gene of interest and an internal reference gene ((β-actin) that provides a normalization factor for the amount of RNA isolated from a specimen.

Paraffin embedded tumor blocks were used for immunhistochemistry. EGFR immunnoreactivity was investigated using the EGFR pharmDx™ (DakoCytomation, Glostrup, Denmark). The intensity of membranous immunostaining was defined as: weak (score 1+), moderate (score 2+) or strong (score 3+).

Statistical Analysis

Objective tumor response to cetuximab, toxicity (acne-like rash), and overall survival were the primary endpoints. The overall survival time was calculated as the period from the first day of cetuximab treatment until death from any cause or until the date of the last follow-up, at which point data were censored.

Gene expression values are expressed as ratios between two absolute measurements: the gene of interest and the internal reference gene, β-actin. The associations between gene expression levels and toxicity (Grade 0-1 vs. Grade 2-3) and response to cetuximab (partial response (PR), stable disease (SD), and progressive disease (PD)) were evaluated by non-parametric methods (the Mann-Whiteney for toxicity and the Kruskal-Wallis for response). To assess the associations between the expression level of each gene and overall survival, the expression level was categorized into a low and a high value at optimal cutpoints. The maximal χ² method of Miller and Siegmund and Halpern was adapted to determine which gene expression (optimal cutpoint) best segregated patients into poor- and good-prognosis subgroups (in terms of likelihood of survival). To determine a P-value that could be interpreted as a measure of the strength of the association based on the maximal χ² analysis, 2000 bootstrap-like simulations were used to estimate the distribution of the maximal χ² statistics under the null hypothesis of no association. The corrected p value was calculated as the proportion of the 2000 simulated maximal χ² statistics that was greater than the original maximal χ². Median survival with 95% confidence intervals (Cls) and the Pike estimate of relative risk with 95% Cls based on the log-rank test were used to provide quantitative summaries of the gene expression data.

All reported P values were two sided. All analyses were performed using the SAS statistical package version 8.2 [SAS Institute Inc. S. SAS/STAT® User's Guide, Version 8. Cary, N.C.: SAS Institute Inc., 1999] and Epilog Plus Version 1.0 [Epicenter Software E. Epilog windows user guide and procedures. Pasadena, Calif.: Epicenter Software, 1999].

Results

A total of 39 patients were enrolled in this study, including 21 women and 18 men with a median age of 64 years (range 35-83). 31 (79%) patients were Caucasian, 6 (15%) were Asian and 2 (5%) were Hispanic. All (100%) patients were assessable to associate EGFR expression detected by immunhistochemical staining to parameter of clinical outcome. 34 (87%) patients were assessable to associate gene expression levels of COX-2, CCND1, EGFR, IL-8 and VEGF to response, survival, and toxicity. With a median follow up period of all 39 patients included in this study of 7.1 months (95% confidence interval [Cl], 2.5 to 21.6 months), the median survival time was 5.5 months (95% Cl, 2.7 to 8.7 months). Two (6%) patients had PR, 21 (60%) patients had SD and 12 (34%) patients had PD under treatment with cetuximab, while no patient had complete response and for 4 patients the response was invaluable. Skin reactions were observed in 85% of the 39 patients, where 12 patients (31%) had a grade 1, 20 patients a grade 2 (51%) and 1 patient had a grade 3 (3%) skin reaction (see Table 2).

COX-2 gene expression was quantifiable in 27 (79%) of the 34 samples, CCND1 expression in 31 samples (91%), EGFR expression in 30 samples (88%), IL-8 expression in 33 samples (97%,) and VEGF expression in 31 samples (91%). The median gene expression levels relative to the internal reference gene β-actin of the analyzed genes are listed in Table 6.

TABLE 6
Gene expression levels relative to the internal reference gene
β-actin of the analyzed genes

| | | mRNA expression levels |
|---|---|---|
| | No. of | relative to β-actin × 10⁻³ |
| Gene | Patients | Median (range) |
| Cox-2 | 27 | 0.61 (0.01-7.23) |
| Cyclin D1 | 31 | 8.41 (1.81-19.03) |
| EGFR | 30 | 0.97 (0.46-55.7) |
| IL-8 | 33 | 1.98 (0.01-44.93) |
| VEGF | 31 | 3.93 (0.96-47.53) |

Abbreviations:
Cox-2, cyclooxygenase 2;
CCND1, cyclin D1;
EGFR, epidermal growth factor receptor;
IL-8, interleukin 8;
VEGF, vascular endothelial growth factor

The only factor that showed a significant correlation between response and gene expression levels was VEGF (see Table 7). Patients with a PR had a median gene expression level of 4.63×10⁻³, patients with stable disease 3.76×10⁻³ and patients with progressive disease 6.56×10⁻³ (P=0.038, Kruskal-Wallis test).

TABLE 7
Gene expression levels and clinical outcome (response, toxicity)
in patients with metastatic CRC treated with cetuximab

| | Total | Cox-2 | | EGFR | | IL-8 | | CCND1 | | VEGF | |
|---|---|---|---|---|---|---|---|---|---|---|---|
| | No. of | | Median | | Median | | Median | | Median | | Median |
| | Patients | n | (range) ×10⁻³ | n | (range) ×10⁻³ | n | (range) ×10⁻³ | n | (range) ×10⁻³ | n | (range) ×10⁻³ |
| Response | 39 | | | | | | | | | | |
| PR | 2 | 2 | 0.18 | 2 | 1.72 | 2 | 1.62 | 2 | 10.4 | 2 | 4.63 |
| | | | (0.17-0.19) | | (0.85-2.58) | | (0.56-2.68) | | (9.08-11.7) | | (3.06-6.19) |
| SD | 21 | 16 | 0.66 | 16 | 0.93 | 18 | 1.55 | 16 | 5.93 | 16 | 3.76 |
| | | | (0.01-7.23) | | (0.53-2.09) | | (0.01-44.9) | | (2.72-11.0) | | (0.96-9.49) |

Gene expression cutoff values that best segregated patients into poor- and good prognosis subgroups (in terms of likelihood of surviving) were defined for COX-2, CCND1, EGFR, IL-8 and VEGF by using the maximal χ² method of Miller and Siegmund and Halpern. The log-rank test was used to evaluate the association between gene expression levels and survival for each single gene. Using an EGFR cutoff value of 1.2×10⁻³, 21 patients had a low EGFR expression level and 9 had a high EGFR expression level. The median survival of patients with low EGFR mRNA levels was 7.3 months (95% Cl, 4.4 to 13.5 months) and 2.2 months (95% Cl, 1.7 to 4.5 months) in patients with high EGFR mRNA levels (P=0.09; log rank test) (see Table 9). The association between expression levels of CCND1, EGFR, IL-8 and VEGF, and survival did not show significant results or relevant trends, as shown in Table 8.

TABLE 8
Analysis of Survival in patients with metastatic CRC treated with
single agent cetuximab: Association with mRNA expression levels
(univariate/combined analyses)

| | | Overall Survival | |
|---|---|---|---|
| | | Median, | Relative Risk |
| Factor | No. of Patients | Months (95% CI) | (95% CI) |
| Cox-2 | | | |
| ≦1.20 × 10⁻³ | 21 | 8.5 (4.5, 15.0) | 1 (Reference) |
| >1.20 × 10⁻³ | 6 | 2.2 (1.8, 4.8) | 2.72 (0.97, 7.63) |
| P value | | | 0.14 |
| EGFR | | | |
| ≦1.20 × 10⁻³ | 21 | 7.3 (4.4, 13.5) | 1 (Reference) |
| >1.20 × 10⁻³ | 9 | 2.2 (1.7, 4.5) | 2.83 (1.14, 7.02) |
| P value | | | 0.09 |
| CCND1 | | | |
| ≦1.20 × 10⁻³ | 7 | 2.3 (1.4, 3.4) | 1 (Reference) |
| >1.20 × 10⁻³ | 24 | 5.7 (4.4, 12.0) | 0.63 (0.25, 1.60) |
| P value | | | 0.86 |
| IL-8 | | | |
| ≦1.20 × 10⁻³ | 27 | 5.7 (3.4, 12.1) | 1 (Reference) |
| >1.20 × 10⁻³ | 6 | 2.7 (2.2, 4.8) | 2.41 (0.88, 6.56) |
| P value | | | 0.27 |
| VEGF | | | |
| ≦1.20 × 10⁻³ | 12 | 2.7 (1.7, 5.5) | 1 (Reference) |
| >1.20 × 10⁻³ | 19 | 5.7 (3.4, 13.5) | 0.62 (0.28, 1.37) |
| P value | | | 0.72 |
| Cox-2, EGFR, | | | |
| and IL8 | | | |
| All low | 12 | 13.5 (5.5, 15.9) | 1 (Reference) |
| Any high | 16 | 2.3 (2.1, 4.8) | 3.32 (1.26, 8.76) |
| P value | | | 0.028 |

Abbreviations:
Cox-2, cyclooxygenase 2;
EGFR, epidermal growth factor receptor;
CCND1, cyclin D1;
IL-8, interleukin 8;
VEGF, vascular endothelial growth factor

The combination of expression levels of COX-2, EGFR and IL-8 lower than 1.2×10⁻³ had a median overall survival of 13.5 months (95% Cl, 5.5 to 15.9 months) and patients with high gene expression levels of these three genes had a median overall survival of 2.3 months (95% Cl, 2.1 to 4.8 months) (P=0.028, log-rank test) (see Table 8). In addition the combination of gene expression levels of COX-2, EGFR and IL-8 was an independent prognostic factor after adjusting skin toxicity which was associated with survival. Other combinations of genes did not show a significant relation to survival.

Skin Toxicity

CCND1, IL-8 and VEGF gene expression levels did not show a significant correlation with the grade of skin toxicity. Patients with a grade 2-3 skin reaction had lower intratumoral EGFR gene expression levels compared to patients with a grade 0-1 toxicity, however it did not reach statistical significance (0.85 vs. 1.19×10⁻³, P=0.086). Patients with skin toxicity of higher grade had statistically significant lower gene expression levels of COX-2 (0.27 vs. 1.20, P=0.009).

There was no significant correlation between skin toxicity and response; nevertheless, only 2 out of 39 patients responded, both of them had higher grade skin toxicity. However, patients with a grade 2-3 skin reaction had a significantly longer median progression free survival of 3.3 months (95% Cl, 2.4 to 4. 6 months) compared with patients that had a grade 0-1 toxicity (median 1.3 months; 95% Cl, 1.1 to 2.0 months; P=0.001, log-rank test) and their overall survival was significantly longer (median 7.7 months 95% Cl, 4.4 to 15.0 months vs. median 2.2 months 95%% Cl, 1.8 to 5.7 months; P=0.049, log-rank test).

Immunhistochemical Analysis of EGFR in Tumor Samples

Immunhistochemical analysis of EGFR protein expression in tumor samples demonstrated the following: 23 (59%) had a weak EGFR staining (1+intensity), 11 (28%) patients a moderate (2+intensity) and 5 (13%) patients a strong EGFR staining (3+intensity). There was no association of EGFR staining with response, survival and toxicity. IHC and gene expression levels of EGFR (see Table 9) were compared in thirty patients. This was not considered a significant association.

TABLE 9
Parameters of clinical outcome in relation to the immunhistochemical analysis of EGFR in patients with
metastatic CRC treated with single agent cetuximab

| | | | | | | | Progression-Free | | | |
|---|---|---|---|---|---|---|---|---|---|---|
| | No. | | | | | | survival | | Overall Survival | |
| | of | | | | Toxicity | | Median, | Relative | Median, | Relative |
| Stain- | Pa- | Response | | | Grade | Grade | Mo | Risk | Mo | Risk |
| ing | tients | PR | SD | PD | 0-1 | 2-3 | (95% CI) | (95% CI) | (95% CI) | (95% CI) |
| 1+ | 23 | 1 (5%) | 14 (67%) | 6 (29%) | 12 (52%) | 11 (48%) | 2.4 (1.8, 3.7) | 1 (Reference) | 5.7 (2.3, 10.7) | 1 (Reference) |
| 2-3+ | 16 | 1 (7%) | 7 (50%) | 6 (43%) | 6 (38%) | 10 (62%) | 1.4 (1.1, 3.7) | 1.11 (0.58, 2.12) | 4.8 (2.3, 12.0) | 1.35 (0.65, 2.81) |
| P | | 0.74 | | | 0.52 | | | 0.74 | | 0.36 |
| value | | | | | | | | | | |

Abbreviations:
PR, partial response;
SD, stable disease;
PD, progressive disease

### Example 3

### Molecular Predictors of Irinotecan Efficacy

The purpose of this study was to investigate whether mRNA levels of enzymes involved in 5-FU metabolism (TS, DPD), in CPT-11 metabolism (MDR1, Topoisomerase 1), in angiogenesis (COX-2, EGFR, IL-8, VEGF) and in DNA-repair/drug detoxification (ERCC1, GSTP1) are associated with the clinical outcome of patients with colorectal cancer (CRC) treated with first-line CPT-11/5-FU (CPT-11 based chemotherapy).

Patients

Fifty-four patients with histopathologically confirmed metastatic CRC, who received first-line CPT-11/ 5-FU based treatment, were included in this study of molecular markers and clinical outcome of CPT-II based therapy. Approval for this study was obtained from the Institutional Review Board of the University of Southern California, Keck School of Medicine. Written informed consent for tissue and blood collection to study molecular correlates was obtained.

All 54 patients received a first-line CPT-11/5-FU based chemotherapy. Of these, 31 patients were enrolled in the following clinical trials: 3C-00-4 (12 patients) and 3C-01-4 (19 patients). The other 23 patients, not included in a clinical trial, were all treated at the University of Southern California/Norris Comprehensive Cancer Center (Los Angeles, Calif.) or at the Los Angeles County/University of Southern California Medical Center (LAC/USC). The clinical evaluation and response criteria of all patients in the study are listed below. When comparing the 31 patients who were enrolled in the two clinical trials to the 23 patients who were not enrolled, there were no statistically significant differences in demographic characteristics (age, sex and race) and clinical outcome variables (tumor, response, toxicity, progression-free survival, and overall survival).

Methods

Tumor samples were obtained from the primary colorectal tumor or from a metastatic site at the time of diagnosis, of 33 patients were available. There were no statistically significant differences in demographic characteristics and clinical outcome variables between patients whose tumor samples were available (n=33) and those whose tumor samples were not available (n=21).

CT imaging for response was performed every 6 weeks. In general, responders to therapy were classified as those patients whose tumor burden had decreased by 50% or more for at least 6 weeks. Patients with evaluable but non-measurable disease, whose tumor and all evidence of disease had disappeared, were classified as showing complete response (CR). Responders with anything less than complete response were simply categorized as demonstrating partial response (PR). Non-responders were, likewise, divided into two separate classification groups. The first of these, progressive disease (PD), was defined as a 25% or more increase in tumor burden (compared to the smallest measurement) or the appearance of new lesions. Further, non-responsive patients, who did not progress within the first 12 weeks following the start of CPT-11/5-FU based chemotherapy, were classified as having stable disease (SD).

Paraffin-embedded tumor blocks were reviewed for quality and tumor content by a pathologist. Ten micrometer thick sections were obtained from identified areas with the highest tumor concentration and were then mounted on uncoated glass slides. For histology diagnosis, three representative sections, consisting of the beginning, the middle and the end sections of the tissue, were stained with H&E by the standard method. Before microdissection, sections were deparafinized in xylene for ten minutes and hydrated with 100%, 95%, and finally, 70% ethanol solutions. Sections were then washed in H₂O for 30 seconds, stained with nuclear fast red (NFR, American MasterTech Scientific, Inc., Lodi, Calif.) for 20 seconds, and rinsed again in H₂O for 30 seconds. Finally, samples were dehydrated with 70% ethanol, 95% ethanol, and 100% ethanol solutions for 30 seconds each, followed by xylene again for ten minutes. The slides were then completely air-dried. If the histology of the samples was homogeneous and contained more than 90% tissue of interest, samples were dissected from the slides using a scalpel. All other sections of interest were selectively isolated by laser capture microdissection (P.A.L.M. Microsystem, Leica, Wetzlar, Germany), according to the standard procedure described in Bonner R. F. et al. (1997) 278:1481-1483. The dissected particles of tissue were transferred to a reaction tube containing 400 μL of RNA lysis buffer.

Tissue samples to be extracted were placed in a 0.5 ml thin-wailed tube containing 400 μl of 4M dithiothreitol (DTT)-GITC/sarc (4 M guanidinium isothiocyanate, 50 mM Tris-HC1, pH 7.5, 25 mM EDTA) (Invitrogen; #15577-018). The samples were homogenized and an additional 60 μl of GITC/sarc solution was added. They were heated at 92° C. for 30 minutes and then transferred to a 2 ml centrifuge tube. Fifty μl of 2M sodium acetate was added at pH 4.0, followed by 600 μl of freshly prepared phenol/chloroform/isoamyl alcohol (250:50:1). The tubes were vortexed for 15 seconds, placed on ice for 15 minutes and then centrifuged at 13,000 RPM for eight minutes in a chilled (8° C.) centrifuge. The upper aqueous phase (250-350 μl) was carefully removed and placed in a 1.5 ml centrifuge tube. Glycogen (10 μl) and 300-400 μl of isopropanol were added and the samples vortexed for 10-15 seconds. The tubes were chilled at −20° C. for 30-45 minutes to precipitate the RNA. The samples were then centrifuged at 13,000 RPM for seven minutes in an 8° C. centrifuge. The supernatant was poured off and 500 μl of 75% ethanol was added. The tubes were again centrifuged at 13,000 RPM for six minutes in a chilled (8° C.) centrifuge. The supernatant was then carefully poured off, so as not to disturb the RNA pellet, and the samples were quick-spun for another 15 seconds at 13,000 RPM. The remaining ethanol was removed with a 20 μl pipette, and the samples were left to air-dry for 15 minutes. The pellet was resuspended in 50 μl of 5 mM Tris. And finally, cDNA was prepared as previously described in Lord R. V. et al. (2000) J. Gastrointest. Surg. 4:135-142.

Quantification of COX-2, DPD, EGFR, ERCC1, GSTP1, 11-8, MDR1, Topo 1, TS, VEGF, and an internal reference gene (β-actin) was done using a fluorescence based realtime detection method (ABI PRISM 7900 Sequence detection System (TaqMan®) PerkinElmer (PE) Applied Biosystem, Foster City, Calif., USA). The PCR reaction mixture consisted of 1200 nM of each primer, 200 nM of probe, 0.4 U of AmpliTaq Gold Polymerase, 200 nM each of dATP, dCTP, dGTP, dTTP, 3.5 mM of MgCl₂, and 1× Taqman Buffer A containing a reference dye. The final volume of the reaction mixture was 20 μl (all reagents from PE Applied Biosystems, Foster City, Calif., USA). Cycling conditions were 50° C. for two minutes, 95° C. for ten minutes, followed by 46 cycles of 95° C. for 15 seconds and 60° C. for one minute. The primers and probes used are listed in Table 10 (SEQ ID NOS. 19 through 51).

TABLE 10
Primers and Probes

| | Gen Bank | Forward primer | Reverse Primer | Taqman probe |
|---|---|---|---|---|
| Gene | Accession | (5′-3′) | (5′-3′) | (5′-3′) |
| β-actin | NM_001101 | GAGCGCGGCTACAGCTT | TCCTTAATGTCACGCACGATTT | ACCACCACGGCCGAGCGG |
| Cox-2 | NM_000963 | GCTCAAACATGATGTTTGCATTC | GCTGGCCCTCGCTTATGA | TGCCCAGCACTTCACGCATCAGTT |
| DPD | NM_000110 | AGGACGCAAGGAGGGTTTG | GTCCGCCGAGTCCTTACTGA | CAGTGCCTACAGTCTCGAGTCTGCCAGTG |
| EGFR | NM_005228 | TGCGTCTCTTGCCGGAAT | GGCTCACCCTCCAGAAGCTT | ACGCATTCCCTGCCTCGGCTG |
| ERCC1 | NM_001983 | GGGAATTTGGCGACGTAATTC | GCGGAGGCTGAGGAACAG | CACAGGTGCTCTGGCCCAGCACATA |
| GSTP1 | NM_000852 | CCTGTACCAGTCCAATACCATCCT | TCCTGCTGGTCCTTCCCATA | TCACCTGGGCCGCACCCTTG |
| IL-8 | NM_000584 | CAGCTCTGTGTGAAGGTGCAGTT | GGGTGGAAAGGTTTGGAGTATGTC | TGCACTGACATCTAAGTTCTTTAGCACTCCTTGGC |
| MDR1 | NM_000927 | GTCCCAGGAGCCCATCCT | ACCCGGCTGTTGTCTCCAT | ACTGCAGCATTGCTGAGAACATTGCCT |
| Topo I | NM_003286 | TGTAGCAAAGATGCCAAGGT | TGTTATCATGCCGGACTTCT | CCTTCTCCTCCTCCAGGACATAAGTGGA |
| TS | NM_001071 | GCCTCGGTGTGCCTTTCA | CCCGTGATGTGCGCAAT | TCGCCAGCTACGCCCTGCTCA |
| VEGF | NM_003376 | AGTGGTCCCAGGCTGCAC | TCCATGAACTTCACCACTTCGT | ATGGCAGAAGGAGGAGGGCAGAATCA |

Abbreviations: Cox-2, cyclooxygenase 2; DPD, dihydropyrimidine dehydrogenase; EGFR, epidermal growth factor receptor; ERCC1, excision repair cross-complementing 1; GSTP1, glutathione S-transferase pi; IL-8, interleukin 8; MDR1, multidrug resistance protein 1; Topo I, topoisomerasel,; TS, thymidylate synthase; VEGF, vascular endothelial growth factor

TaqMan® measurements yield Ct values that are inversely proportional to the amount of cDNA in the tube. For example, a higher Ct value means that more PCR cycles are required to reach a certain level of cDNA detection. Gene expression values (relative mRNA levels) are expressed as ratios (differences between the Ct values) between the gene of interest and an internal reference gene (β-actin). This reference gene provides a baseline measurement for the amount of RNA isolated from a specimen.

Response

Tumor response to CPT-11 based therapy was the primary endpoint in this study. Patients with complete response or partial response (tumor burden decreased by ≧50%) were classified as responders, while patients with stable disease or progressive disease were classified as non-responders. Progression-free survival, toxicity, and overall survival were the secondary endpoints. The progression-free survival time was calculated as the period from the first day of CPT-11 based treatment until the first observation of disease progression or death from any cause. If a patient had not progressed or died, progression-free survival was censored at the time of the last follow-up. Thus the overall survival time was calculated as the time from the first day of CPT-11 based treatment until death from any cause, or until the date of the last follow-up.

Gene expression values are expressed as ratios between two absolute measurements, that of the gene of interest and that of the internal reference gene, β-actin. The associations between gene expression levels, response to CPT-11 based therapy (responders vs. non-responders). and toxicity (Grade 0-2 vs. Grade 3-4) were evaluated by non-parametric methods (the Mann-Whitney U test). In addition, a classification and regression tree (CART) method, based on recursive partitioning (RP), was used to explore gene expression variables for identifying homogenous subgroups for tumor response to CPT-11 based therapy.

The RP analysis is a nonparametric statistical method for modeling a response variable and multiple predictors. The PR analysis includes two essential processes: tree-growing and tree pruning. The tree-growing procedure starts with all patients in one group and makes a series of binary splits, which are based on gene expression variables that define the most distinct subgroups of tumor response. At each partitioning, the tree-growing method examines every possible split for all gene expression variables, in order to select the best cut point. In case of missingness in the primary splitting gene expression variable, the patients are classified based on alternative splits (surrogates). The process is repeated until the terminal nodes reach a minimum size (n=5). The splitting rule of RP is based on the Gini diversity index (one minus the sum of squared probabilities over all levels of response). Once the tree-growing procedure is completed, the tree-pruning process begins to produce a sequence of simpler sub-trees, as misclassification errors associated with a particular sub-tree are assessed. The goal of tree-pruning is to select a final tree from the collection of sub-trees, which minimizes both the relative cost (a measure of the misclassification error) and the number of terminal nodes. This RP analysis included all patients with tumor tissue specimen who were evaluable for tumor response (n=32).

To assess the associations between the expression level of each gene and progression-free survival or overall survival, the expression level was categorized into a low and a high value at optimal cutpoints. The maximal χ² method of Miller and Siegmund (Biometrics 38:1011-1016) and Halpern (Biometrics 38:1017-1023) was used to determine which gene expression (optimal cutpoint) best segregated patients into poor- and good-prognosis subgroups (in terms of likelihood of progression-free survival). To determine a P-value that could be interpreted as a measure of the strength of the association based on the maximal χ² analysis, 2000 bootstrap-like simulations were used to estimate the distribution of the maximal χ² statistics under the null hypothesis of no association. The corrected P-value was calculated as the proportion of the 2000 simulated maximal χ² statistics that was greater than the original maximal χ^{2.} The Pike estimate of relative risk with 95% Cls based on the log-rank test were used to provide quantitative summaries of the gene expression data.

All reported P-values were two-sided. All analyses were performed using the SAS statistical package version 8.2 [SAS Institute Inc. S.SAS/STAT® User's Guide, Version 8. Cary, N.C.: SAS Institute Inc., 1999] and CART 5.0 [San Diego, Calif.: Salford Systems, 1995].

There were 16 female and 17 male patients with a median age of 53 (range 40-75) in this study. Fifteen (45%) were Caucasian, eight (24%) were Hispanic, seven (21%) were Asian, and three (9%) were African-American. All patients were assessable to associate gene expression levels of COX-2, DPD, EGFR, ERCC1, GSTP1, IL-8, MDR1, Topo I, TS, and VEGF with response, progression-free survival/overall survival, and toxicity. The median progression-free survival was 9.9 months (95% Cl, 7.0 to 11.8 months), and the median overall survival time was 27.9 months (95% Cl, 21.3 to 56.6+months) with median follow up of 27.4 months (range: 18.7 to 56.6 months). Seventeen patients were dead. One patient showed complete response (CR) (3%), 12 (38%) patients showed partial response (PR), 13 (41%) patients had SD, and 6 (19%) patients had PD under treatment with 1^{st}-line CPT-based chemotherapy. For one patient, the response was inevaluable. Gastrointestinal side effects/toxicity was observed in 32% of the 33 patients, where 15 (47%) patients had Grade I-Il toxicity and 17 (53%) patients had Grade III-IV toxicity (see Table 11).

TABLE 11
Demographic and clinical parameters of patients with
metastatic CRC treated with first-line CPT-11/5-FU

| Characteristics | Frequency | % |
|---|---|---|
| Median age, years (range) | 53 (40-75) | |
| Gender | | |
| Female | 16 | 48% |
| Male | 17 | 52% |
| Race | | |
| Asian | 7 | 21% |
| Black | 3 | 9% |
| Caucasian | 15 | 45% |
| Hispanic | 8 | 24% |
| Response | | |
| Complete response | 1 | 3% |
| Partial response | 12 | 38% |
| Stable disease | 13 | 41% |
| Progressive disease | 6 | 19% |
| Inevaluable/early off | 1 | |
| study | | |
| Toxicity | | |
| Grade 0-2 | 15 | 47% |
| Grade 3-4 | 17 | 53% |
| Inevaluable/early off | 1 | |
| study | | |

Gene Expression Levels of COX-2, DPD, EGFR, ERCC1, GSTP1, IL-8, MDR1, Topo I, TS and VEGF

COX-2 gene expression was quantifiable in 25 (76%) of the 33 samples, expression of DPD expression in 31 samples (94%), expression of EGFR, MDR-1 and TS- in 32 samples (97%), ERCC1 expression in 33 samples (100%), GSTP1 expression in 33 samples (100%), IL-8 expression in 33 samples (100%), MDR-I expression in 32 samples (97%), Topo I expression in 33 samples (100%), TS in 32 samples (97%), and VEGF expression in 33 samples (100%). The reason for the difference in the number of samples with quantifiable gene expression levels is due to the low or limited amount of cDNA/RNA generated from the microdissected paraffin-embedded tissues. The median gene expression levels, relative to the internal reference gene β-actin), of the analyzed genes are listed in Table 12.

TABLE 12
Gene expression levels relative to the internal reference gene
β-actin of the analyzed genes

| | | mRNA expression levels |
|---|---|---|
| | | relative to β-actin × 10⁻³ |
| Gene | No. of Patients | Median (range) |
| TS | 32 | 1.48 (0.39-5.64) |
| DPD | 31 | 0.31 (0.11-0.95) |
| MDR-1 | 32 | 0.93 (0.12-12.67) |
| Topo I | 33 | 2.11 (0.66-5.73) |
| Cox-2 | 25 | 0.40 (0.07-3.99) |
| EGFR | 32 | 1.03 (0.58-2.37) |
| IL-8 | 33 | 2.96 (0.28-62.50) |
| VEGF | 33 | 3.82 (1.79-17.87) |
| ERCC1 | 33 | 0.42 (0.18-0.98) |
| GSTP-1 | 33 | 2.28 (0.37-8.64) |

Abbreviations:
Cox-2, cyclooxygenase 2;
DPD, dihydropyrimidine dehydrogenase;
EGFR, epidermal growth factor receptor;
ERCC1, excision repair cross-complementing 1;
GSTP1, glutathione S-transferase pi;
IL-8, interleukin 8;
MDR1, multidrug resistance protein 1;
Topo I, topoisomerasel,;
TS, thymidylate synthase;
VEGF, vascular endothelial growth factor

Gene Expression Levels and Response of Patients Receiving 1st˜line CPT-11 Based Chemotherapy

High intratumoral mRNA levels of EGFR, ERCC1, GSPTP1, and MDR1 were each significantly associated with response to CPT-11 based chemotherapy (p≦0.05; Mann-Whitney U test). Ten gene expression variables were considered for the RP analysis. Of the ten gene expression variables evaluated, the RP analysis identified EGFR expression level as the best single split with the best cutoff (≦1.58×10⁻³) between responders and non-responders. The next best split in the lower EGFR group was ERCC1 expression level with the best cutoff of 0.58×10⁻³. Among patients with higher EGFR levels, no further splits could be identified. There were 17, 5, and 10 patients in the three terminal nodes I, II, and III, respectively. Group I (EGFR level ≦1.58×10⁻³ and ERCC1≦0.58×10⁻³) was classified as a group of nonresponders (zero of 17 patients responded to treatment), and Groups II (EGFR level ≦1.58×10⁻³ and ERCC1>0.58×10⁻³) and III (EGFR level >1.58×10⁻³) were classified as groups of responders (13 of 15 patients responded to treatment).

Gene Expression Levels and Progression-free Survival/Overall Survival in Patients Receiving 1^{st}-line CPT-11 Based Chemotherapy

Expression levels of single genes analyzed in this study did not show significant correlations with progression-free survival nor overall survival in the univariate analysis.

Patients who were classified as responders by the RP analysis (Groups II and III) were at a lower risk for progression (relative risk=0.48, 95% Cl: 0.22-1.05), compared to patients who were classified as non-responders by the RP analysis (Group I) (log-rank test p=0.038). Patients in Groups II and III also were at lower risk for dying (relative risk=0.46, 95% Cl: 0.17-1.27) compared to Group I patients (log-rank test p=0.12).

Gene Expression Levels and Gastrointestinal Toxicity in Patients Receiving 1^{st}-line CPT-11 Based Chemotherapy

Expression levels of genes analyzed in this study did not show significant correlations with the grade of gastrointestinal toxicity.

Correlation of Gene Expression Levels of EGFR and Other Factors Analyzed: Based on the results of the recursive partitioning analysis, which described EGFR as the most important factor influencing the clinical outcome of patients receiving CPT-11 based chemotherapy, the correlation between EGFR and other genes analyzed in this study was reviewed. As shown in Table 12, the mRNA levels of EGFR had a statistically significant correlation with ERCC1, GSTP1, MDR1, and VEGF (Spearman correlation coefficients ≧0.4; p<0.05, Table 13).

TABLE 13
Relationship (Spearman Correlation Coefficients) among gene
expression levels of genes analyzed in this study

| | EGFR |
|---|---|
| Cox-2 | −0.04 |
| DPD | −0.001 |
| ERCC1 | 0.40* |
| GSTP-1 | 0.42* |
| IL-8 | 0.07 |
| MDR-1 | 0.48** |
| Topo I | 0.33 |
| TS | 0.09 |
| VEGF | 0.43* |

p < 0.05;
**p < 0.01
Abbreviations:
Cox-2, cyclooxygenase 2;
DPD, dihydropyrimidine dehydrogenase;
EGFR, epidermal growth factor receptor;
ERCC1, excision repair cross-complementing 1;
GSTP1, glutathione S-transferase pi;
IL-8, interleukin 8;
MDR1, multidrug resistance protein 1;
Topo I, topoisomerasel,;
TS, thymidylate synthase;
VEGF, vascular endothelial growth factor

Discussion

The use of CPT-11 in combination with 5-FU has significantly improved the clinical outcome of patients with metastatic CRC. Douillard, J. Y. et al. (2000) Lancet 355:1041-1047 and Water, J. and Cunningham, D. (2001) Br. J. Cancer 84:1-7. However, there are currently no molecular markers established to identify patients who will most likely benefit from the CPT-11 based chemotherapy. One goal of this study was to identify gene expression levels of enzymes involved in critical pathways of cancer progression to predict response, survival/time to tumor progression, and toxicity in patients undergoing first line CPT-11 based chemotherapy.

The results of the few reported studies which have tried to characterize factors determining the clinical outcome of patients receiving CPT-11-based therapy are controversial. In a pilot study of 11 patients with metastatic CRC, Saltz et al. suggested that gene expression levels of Topo 1 and TS are predictors for responsiveness to CPT-11. Saltz, L. et al. (1998) Proc. Am. Soc. Clin. Oncol. 17:991 (abstract). In contrast, Paradiso et al. (2004) Int. J. Cancer 111:252-258) failed to show any significant correlation between protein levels of Topo 1 and TS and the clinical outcome in 62 patients with advanced CRC. One possible reason for these discrepant results is that different detection methods were used to measure TS and Topo 1. While Saltz et al. used quantitative Real-Time PCR to detect gene expression levels, Paradiso et al. used immunhistochemistry (IHC) to detect protein levels of Topo-1 and TS. IHC is a semiquantitative and subjective method, and it is limited by the sensitivity of the monoclonal antibody used and the necessity for tissue handling. Another possible explanation for the discrepant results between the two studies is simply the fact that Saltz et al. only included 11 patients in their pilot-study. There is yet to be validated in a larger prospective clinical trial.

High intratumoral gene expression levels of EGFR, ERCC-1, MDR-1, DPD and GST-P1 were associated with response to first line CPT-11 based chemotherapy in 33 patients with metastatic CRC. Using a recursive partitioning analysis, EGFR was shown to be the most important factor among all analyzed genes in distinguishing responders from non-responders treated with CPT-11 based chemotherapy. HIHG EGFR expression has been correlated with various different cellular processes involved in carcinogenesis, such as cell proliferation, inhibition of apoptosis, angiogenesis, cell motility, and metastasis. Mendelsohn, J. et al. (2000) 19:6550-6565 and Herbst, R. S. and Shin, D. M. (2002) Cancer 94:1593-1611.

This study shows that mRNA levels of EGFR had a statistically significant correlation with ERCC1 gene expression, an enzyme involved in the DNA-repair pathway. Moreover, high ERCC1 gene expression levels were significantly associated with response to CPT-11 based chemotherapy in patients with metastatic CRC. Behind EGFR, ERCC1 ranked as the next most influential factor in distinguishing responders from non-responders. In addition, the combination of high expression levels of both genes showed a significant longer progression-free survival of longer duration. ERCC1 is an important member of the nucleotide excision repair system, known to be involved in the repair of DNA damage caused by platinum agents. Prewett, M. C. et al. (2002) Clin. Cancer Res. 8:994-1003.

Several studies have shown that high intratumoral ERCC-1 gene expression levels lead to a higher chemoresistance to platinum based chemotherapy in patients with gastrointestinal tumors. The primary hypothesis was that tumors with high levels of ERCC-1 would be more resistant to CPT-11 based chemotherapy, instead, these findings demonstrate the opposite. In vitro studies by Yacoub et al. ((2003) Radiat. Res. 159:439-452) using prostate cancer cells showed that epidermal growth factor markedly increased ERCC1 expression levels through the mitogen-activated protein kinase pathway. This data shows a significant correlation between EGFR and ERCC1 indicating that EGFR may be upstream of the regulation of DNA repair enzymes such as ERCC-1. High levels of ERCC-1 may be an indicator of increased DNA repair reflecting tumor cells requiring a high base line of DNA repair due to ongoing mistakes during replication. This instable tumor genome with high activity of replication, DNA repair, high activity of helicases could be responsible for making it these cells more vulnerable to topoisomerase inhibitors such as CPT-11.

In this study, patients with high mRNA levels of GSTP-1 were also significantly associated with response to CPT-11 based chemotherapy and GST-PI was significantly correlated with EGFR expression. GSTP-1 is a member of the Glutathione S-transferases (GSTs), a superfamily of metabolic enzymes, which play an important role in the cellular defense system. These enzymes catalyze the conjugation of toxic and carcinogenic molecules with glutathione, thereby protecting cellular macromolecules from damage. It was expected that high expression levels of GSTP-1 in this study would correlate with response to CPT-11-based chemotherapy. Patients with high intratumoral GSTP-1 gene expression levels were associated with response to CPT-11 based chemotherapy. This suggests that EGFR may be involved in the regulation of GSTP-1. Consequently, without being bound by any theory, it is reasonable to speculate that the significant association of high GSTP-1 mRNA levels with response is primarily due to up-regulated EGFR, expression levels. This speculation is strengthened by the fact that GSTP-1, itself, seems to be less important in response prediction of CPT-11-based chemotherapy compared to EGFR and ERCC1, as shown in the recursive partitioning analysis.

MDR1 (synonyms: ABCB1, P-glycoprotein 1) belongs to the superfamily of ABC transporters, which are membrane localized drug-pumps that facilitate cellular efflux mechanisms. Evidence suggests that MDR1 plays a major role in the biliary excretion of CPT-11. Chu, X. Y. et al. (1999) Drug. Metab. Dispos. 27:440-441 and Iyer, L. et al. (2002) Cancer Chemother. Pharmacol. (2002) 49:336-341. lyer et al. (2002), supra, showed that in normal versus MDR1 -deficient mice, the biliary excretion of CPT-11 significantly decreases in MDR1 -deficient mice. This suggests that this transporter contributes to the elimination of CPT-11 by mediating its direct secretion from the blood into the intestinal lumen. However, in this study, patients with high intratumoral gene expression levels of MDR1 were significantly associated with greater response to CPT-11 based chemotherapy.

For interpretation of the data, CART modelling was used because of its potential to discover the pattern of gene expression levels associated with response to CPT-11 based chemotherapy when considering all candidate genes. The CART model has advantages over traditional multivariate regression methods (i.e., logistic regression) that are used to model one response variable and multiple predictors (Cook, (2004) Stat. Med. 23(9):1439 and Fonarow, (2005) JAMA 293(5):572). CART overcomes sample size limitation, makes no prior assumptions of the underlying distribution of predictors, constructs and internally validates the model more efficiently compared to the traditional model. As the traditional regression techniques, the findings from the CART model need independent validation.

### Example 4

### Survival Differences Related to Estrogen Receptor Beta (ERβ) Polymorphism and Age in Female Patients with Metastatic Colon Cancer

Estrogen replacement therapy decreases the risk of colon cancer in postmenopausal women. ERβ, not ERα, is expressed in the colon. ERβ function in colon tissue has been linked with tumor development, apoptosis and prognostic markers. ERβ gene contains a polymorphic dinucleotide CA repeat in the 3′ noncoding region which is associated with hormone levels in females. Thus, this study analyzed samples isolated from metastatic colon cancer patients to determine: 1) whether ERβ polymorphism are associated with survival, and 2) if a difference in survival between young (pre-menopausal) and old (post-menopausal) patients exists.

Patients

A population of 388 patients with metastatic colon cancer collected from 1997 to 2003 were selected for by age. The age extremes were selected because estrogen levels were important for this study and menopause will affect hormone levels. 29 patients of young age (<40 years) were identified, 17 pre-menopausal females and 12 males. In the older population (>65 years) there were 82 patients, 33 post-menopausal females and 49 males.

Methods

Genomic DNA from blood or paraffin embedded tissue was analyzed by 5′-end ³³P-rATP labeled PCR to assess the number of CA repeats in the ERβ gene.

Results

Women with one or more alleles containing ≦18 CA repeats had a 32.1 month median survival while those with both alleles >18 CA repeats had a 19.4 month median survival (p=0.007). Women with both alleles >18 CA repeats had a relative risk of dying 3.12 times that of the other group. There was a significant difference in overall survival among women less than 40 years of age (median survival, 18.7 months) compared with women over 65 years of age (median survival, 30.7 months) (p=0.014), suggesting that younger women have a more aggressive form of disease.

Thus, this study shows that women with metastatic colon cancer that have at least one allele with ≦18 CA repeats have better overall survival than those with both alleles >18 CA repeats. Women with metastatic colon cancer under age 40 had a lower overall survival than women with metastatic colon cancer over age 65.

Thus, this invention provides a method for predicting disease aggression in a female metastatic colorectal cancer patient who is under 40 years of age, the method comprising screening a suitable cell or tissue sample isolated from the patient and detecting the presence and number of CA base pair repeats in the 3-noncoding region of the estrogen receptor β. A therapeutic regimen to combat the aggressiveness of the cancer can then be considered for each patient.

### Example 5

### Gene Expression and Clinical Outcome in Patients with GI Malignancies

Previous studies in patients with colorectal cancer have shown that elevated intratumoral expression levels of a number of genes involved in fluoropyridimine metabolism, including thymidylate synthase (TS), and dihydropyrimidine dehydrogenase (DPD), are associated with poor response to 5-FU-based treatment. Moreover, intratumoral epidermal growth factor receptor (EGFR) overexpression has been associated with resistance to neoadjuvant radiotherapy in rectal cancer. The role of VEGF in angiogenesis, tumor metastasis, and clinical outcome has been demonstrated. In addition, experiments in human xenografts also suggest that vascular endothelial growth factor (VEGF) overexpression could protect cells from the cytotoxic effects of ionizing radiation. DNA repair enzymes Rad5l and excision-repair cross-complementing group 1 (ERCC-1) have been shown to have a role in radiation sensitivity. However, the role of genetic markers in the selection of adjuvant treatment of rectal cancer patients remains unclear.

This study analyzed mRNA levels of 6 putative prognostic or predictive markers for clinical outcome to chemoradiotherapy. Gene expression levels in carcinoma cells and also tumor-adjacent normal rectal tissue using laser capture microdisection were analyzed. Gene expression was analyzed for TS, DPD, ERCC-1, RAD51, VEGF, and EGFR.

Patients

Sixty-seven patients with locally advanced rectal cancer who were treated with adjuvant chemoradiotherapy were eligible for the current study. Forty-three patients were treated at the University of Southern California/Norris Comprehensive Cancer Center (USC/NCCC) or the Los Angeles County/University of Southern California Medical Center (LAC/USCMC) between 1991 and 2000. Twenty-four patients who were treated at outside facilities were referred to USC/NCCC or LAC/USCMC either after their recurrence or for routine follow-up. Patients underwent lower anterior resection (LAR; n=40), abdominal perineal resection (APR; n=19), or transanal resection (TR; n=8), followed by 5-FU infusion plus pelvic radiation. Pelvic irradiation was given as a dose of 45 Gy to the whole pelvis and an additional boost up to 54 Gy (range 50.4-54). During radiation, patients received 5-FU either as a 4-day infusion (1000 mg/M²) at the beginning and end of radiation treatment or as a daily continuous infusion (200 mg/m²).

Patient data were collected retrospectively through chart review. Informed consent was signed by all patients involved in the study and the study has been approved by the Investigational Review Board.

Methods

Samples for gene expression analysis were obtained during the surgical procedure. All samples were formalin-fixed and paraffin-embedded. Sections of 10 μm thickness were taken from the blocks of tumor tissue. Every 4th section was routinely stained with hematoxylin and eosin and evaluated by a pathologist.

All paraffin embedded specimens underwent laser-capture-microdissection in order to isolate RNA from tumor tissue. [P.A.L.M. Microsystem, Leica, Wetzlar, Germany]. Tumor specimens contained cancer cells only (>90% of the microdissected cells were tumor cells). Normal specimens were obtained from the same slide as the tumor sample in maximal distance from the tumor. Areas with normal tissue were isolated by micro-dissection with a scalpel and contained a combination of benign epithelium and stroma. RNA isolation after dissection was done according to a proprietary procedure (U.S. Pat. No. 6,248,535). Following RNA isolation, cDNA was prepared from each sample as described in Lord, R V et. al. (2000) J. Gast. Surg. 4:135.

RNA isolation from paraffin embedded tissue was successful in 55 of 67 tumor specimens and in 47 adjacent normal specimens. In the remaining samples, analysis was not possible because either: 1) the tissue sample contained only tumor but no normal tissue (n=10), 2) the sample contained only normal tissue but no tumor (n=2), 3) RNA could be isolated neither from tumor nor from normal tissue due to degradation or insufficient amount of RNA in the paraffin-embedded samples (n=10).

Quantification of the genes of interest and an internal reference gene (beta [β]-actin) was conducted using a fluorescence-based real-time detection method (ABI PRISM 7900 Sequence Detection System [TaqMan®]; Perkin-Elmer Applied Biosystems, Foster City, Calif.), as previously described in Gibson U E et. al. (1996) Genome Res. 6:995 and Heid, Calif. (1996) Genome Res. 6:986. The PCR mixture consisted of 600 nmol/L of each primer, 200 nmol/L probe (sequences used are given in Table 16.), 5 units of AmpliTaq® Gold polymerase, 200 μmol/L each of dATP, dCTP, dGTP, and dTTP, 3.5 mmol/L MgCl2, and 1×TaqMan® buffer A, containing a reference dye, to a final volume of 20 μL (all reagents were supplied by Perkin-Elmer Applied Biosystems). Cycling conditions were 50° C. for 10 seconds and 95° C. for 10 minutes, followed by 46 cycles at 95° C. for 15 seconds and 60° C. for 1 minute. Colon, liver, and lung RNAs (all Stratagene, La Jolla, Calif.) were used as control calibrators on each plate. Primers and probes are identified in Table 14.

TABLE 14

Primer and probe sequences
of the analyzed genes.

| Gene | Sequences |
|---|---|
| TS | |
| Forward primer | 5′-GCCTCGGTGTGCCTTTCA-3′ |
| Reverse primer | 5′-CCCGTGATGTGCGCAAT-3′ |
| Probe | 6FAM-5′-TCGCCAGCTACGCCCTGCTCA-3′- |
| | TAMRA |
| DPD | |
| Forward primer | 5′-TCACTGGCAGACTCGAGACTGT-3′ |
| Reverse primer | 5′-TGGCCGAAGTGGAACACA-3′ |
| Probe | 6FAM-5′-CCGCCGAGTCCTTACTGAGCACAGG- |
| | 3′-TAMRA |
| ERCC1 | |
| Forward primer | 5′-GGGAATTTGGCGACGTAATTC-3′ |
| Reverse primer | 5′-GCGGAGGCTGAGGAACAG-3′ |
| Probe | 6FAM-5′-CACAGGTGCTCTGGCCCAGCACATA- |
| | 3′-TAMRA |
| RAD51 | |
| Forward primer | 5′-AGGTGAAGGAAAGGCCATGTAC-3′ |
| Reverse primer | 5′-CATATGCTACATTATCCAGGACATCA-3′ |
| Probe | 6FAM-5′-TGCCAGAGAGACCATACCTCTCAGCCA- |
| | 3′-TAMRA |
| VEGF | |
| Forward primer | 5′-AGTGGTCCCAGGCTGCAC-3′ |
| Reverse primer | 5′-TCCATGAACTTCACCACTTCGT-3′ |
| Probe | 6FAM-5′-ATGGCAGAAGGAGGAGGGCAGAATCA- |
| | 3′-TAMRA |
| EGFR | |
| Forward primer | 5′-TGCGTCTCTTGCCGGAAT-3′ |
| Reverse primer | 5′-GGCTCACCCTCCAGAAGCTT-3′ |
| Probe | 6FAM-5′-ACGCATTCCCTGCCTCGGCTG-3′- |
| | TAMRA |
| β-Actin | |
| Forward primer | 5′-TGAGCGCGGCTACAGCTT-3′ |
| Reverse primer | 5′-TCCTTAATGTCACGCACGATTT-3′ |
| Probe | 6FAM-5′-ACCACCACGGCCGAGCGG-3′-TAMRA |

Statistical Analysis

Recurrence status was categorized into the following groups: (1) had pelvic recurrence or distant metastases within 5 years since completion of adjuvant chemoradiation; (2) did not have developed pelvic recurrence nor distant metastases within 5 years since completion of adjuvant chemoradiation. The associations of recurrence with patient characteristics including demographic (age, sex, and race), and pretreatment information (grade, T-stage, N-stage, and type of surgery) were summarized using a contingency table and were formally tested by Fisher's exact tests.

QRT-PCR analyses yield values that are expressed as ratios between two absolute measurements: the gene of interest and the internal reference gene, b-actin. All gene expression levels were log-transformed prior to analysis. In order to compare gene expression levels between tumor and tumor-adjacent tissue, two different approaches were used: 1) To test whether tumor tissue expressed on average higher/lower mRNA levels than corresponding normal tissue, a 2-sided paired t test was used. Box-plots displayed the differences in gene expression levels between normal and tumor tissue. 2) To evaluate whether within the same patient tumor tissue with higher expression levels is found to have tumor-adjacent tissue also with higher expression levels, the Pearson correlation coefficient was calculated.

The association between each gene expression variable and recurrence was evaluated by in the initial univariate analysis. Secondly, to assess the associations between the expression levels of genes in each pathway and recurrence, plots of mRNA expression levels of two genes by recurrences status were generated to visualize the associations. The expression level of each gene was then categorized into a low and a high value at optimal cutpoints using the maximal χ² method of Miller and Siegmund and Halpern. Patients were classified into two groups: (1) low mRNA levels of two genes in the pathway; (2) high mRNA levels of any of two genes in the pathway. To determine a P-value that could be interpreted as a measure of the strength of the association between a combination of gene expression in each pathway and recurrence status based on the optimal cutpoint approach, 2000 bootstrap-like simulations were used to estimate the distribution of the maximal χ² statistics under the null hypothesis of no association. The corrected p value was calculated as the proportion of the 2000 simulated maximal χ² statistics that was greater than the original maximal χ².

Finally, a classification and regression tree (CART) method based on recursive partitioning (RP) was used to explore gene expression variables for identifying homogenous subgroups for recurrence after completion of adjuvant. The RP analysis is a nonparametric statistical method for modeling a response variable and multiple predictors. The PR analysis includes two essential processes: tree-growing and tree pruning. The tree-growing procedures starts with all patients in one group and makes a series of binary splits based on predictors that defined the subgroups most distinct in tumor recurrence. At each partitioning, the tree-growing method examines all possible splits for all gene expression variables and baseline variables to select the best cut point. In case of missingness in the primary splitting gene expression variable, the patients are classified based on alternative splits (surrogates). The process is repeated until the terminal nodes reach a minimum size (n=5). The splitting rule of RP is based on the Gini diversity index (1—the sum of squared probabilities over all levels of response). After the tree-growing procedure have completed, the tree-pruning process starts to produce a sequence of simpler subtrees through assessing the misclassification error associated with a particular subtree. The goal of tree-pruning is to select a final tree from the set of subtrees that minimize both the relative cost, a measure of the misclassification error, and the number of terminal nodes. The RP analysis included all patients with any gene mRNA levels available (n=57).

All reported P values were two-sided. All analyses were performed using the SAS statistical package version 8.2 [SAS Institute Inc. S. SAS/STAT® User's Guide, Version 8. Cary, N.C.: SAS Institute Inc., 1999], and CART 5.0 (Steinberg D, and Colla P. CART: Tree-Structured Non-Parametric Data Analysis. San Diego, Calif.: Salford Systems, 1995).

Results

This study cohort consisted of 25 (37%) women and 42 (63%) men with a median age of 52 years (range 25 to 79 years). In terms of ethnic background, 47 patients were white, 13 Hispanic, 5 Asian, and 2 African-American. Histological staging revealed 19 patients to be stage T2 and 48 patients to be stage T3. Twenty-four patients had no involvement of regional lymph nodes (pN₀), 35 had ≧1 lymph node metastasis (pN₊), and lymph node status of 8 patients who received transanal resection was not assessable. The tumors were graded histopathologically as highly differentiated (1 patient) moderately differentiated (55 patients), and poorly differentiated (11 patients). No patient had systemic metastases at the time of first diagnosis.

Thirty-four patients developed pelvic tumor recurrence or distant metastases within 5 years of completion of adjuvant chemoradiation, 33 patients did not develop recurrence nor did metastases within 5 years of completion of adjuvant chemoradiation.

The comparison analysis among patients with complete data vs. those with partial or no gene expression data, there was no statistically significant association between recurrence and missingness of gene expression data (Wilcoxon test, P=O.99).

There was no significant association between demographical (sex, age, ethnicity) or clinical (T-stage, N-stage, grade of differentiation and type of surgery) variables and recurrence status (the Fisher's exact test, see Table 17). However, females in this cohort did show a trend toward decreased risk of recurrence. See Table 15.

TABLE 15
Recurrence in rectal cancer based on demographic and clinical
parameters

| Parameter | n | Recurrence-free | Recurrence | P value* |
|---|---|---|---|---|
| Age, years | | | | 1.00 |
| <50 | 27 | 13 (48%) | 14 (52%) | |
| ≧50 | 40 | 20 (50%) | 20 (50%) | |
| Sex | | | | 0.08 |
| Male | 42 | 17 (40%) | 25 (60%) | |
| Female | 25 | 16 (64%) | 9 (36%) | |
| Ethnicity | | | | 0.11 |
| White | 47 | 20 (43%) | 27 (57%) | |
| Other | 20 | 13 (65%) | 7 (35%) | |
| pT | | | | 0.59 |
| pT2 | 19 | 8 (42%) | 11 (58%) | |
| pT3 | 48 | 25 (52%) | 23 (48%) | |
| pN | | | | 0.80 |
| pN0 | 24 | 12 (50%) | 12 (50%) | |
| pN+ | 35 | 16 (46%) | 19 (54%) | |
| Grade | | | | 1.00 |
| I-II | 56 | 28 (50%) | 28 (50%) | |
| III | 11 | 5 (45%) | 6 (55%) | |
| Surgery Typea | | | | 0.60 |
| APR | 19 | 8 (42%) | 11 (58%) | |
| LAR | 40 | 20 (50%) | 20 (50%) | |
| TR | 8 | 5 (63%) | 3 (38%) | |

*Based on the Fisher's exact test
a. APR, abdominal perineal resection; LAR, lower anterior resection; TR, transanal resection

A significant association between mRNA expression in tumor and corresponding tumor-adjacent samples for the genes ERCC-1 (Pearson Correlation Coefficient R=0.56, P<0.001) and VEGF (R=0.52, P<0.001), moderate association for the genes of TS (R=0.30, P=0.06) and DPD (R=0.32, P=0.07), but not for EGFR (R=0.03, P=0.87) and RAD51 (R=0.14, P=0.39) was observed.

There were significant differences in mRNA expression levels for DPD and VEGF between tumor tissue and tumor-adjacent normal tissue (Table 18). The expression level of DPD was statistically significantly lower in tumor tissue compared to that in tumor-adjacent normal tissue (p<0.01). The expression level of VEGF was statistically significantly higher in tumor tissue compared to that in tumor-adjacent normal tissue (p<0.001). See Table 16.

TABLE 16
mRNA expression values for the analyzed genes in tumor and
tumor-adjacent tissue

| | Tumor-adjacent | | | | | | |
|---|---|---|---|---|---|---|---|
| | normal tissue | | | Tumor tissue | | | |
| | | Geo- | | | Geo- | | |
| | | metric | | | metric | | P |
| Parameter | N | mean | 95% CI | N | mean | 95% CI | value* |
| TS | 45 | 1.75 | 1.51-2.01 | 53 | 1.76 | 1.43-2.13 | 0.89 |
| DPD | 40 | 0.93 | 0.76-1.12 | 47 | 0.62 | 0.53-0.71 | 0.004 |
| RAD51 | 42 | 0.94 | 0.63-1.31 | 50 | 1.05 | 0.77-1.37 | 0.92 |
| EGFR | 43 | 0.87 | 0.68-1.09 | 49 | 0.64 | 0.47-0.82 | 0.13 |
| ERCC1 | 46 | 1.44 | 1.20-1.71 | 52 | 1.48 | 1.23-1.77 | 0.50 |
| VEGF | 4 | 1.77 | 1.48-2.11 | 52 | 5.10 | 4.26-6.06 | <0.001 |

*Based on the paired Student-T tests

Gene Expression and Recurrence

The expression levels of genes in tumor-adjacent tissue and tumor tissue by local recurrence were summarized in Table 15. The gene expression levels of TS, DPD, EGFR, ERCC-1, and VEGF in tumor-adjacent normal tissue were higher in patients who developed early recurrence than those in patients who did not developed early recurrence, with TS, EGFR, and VEGF reaching statistical significance. However, mRNA levels of TS, DPD, ERCC1, and EGFR in tumor tissue were not different in patients across recurrence groups. Only intratumoral VEGF mRNA level was statistically higher in patients who had early recurrence than that in patients without early recurrence. A combination of mRNA expression levels of two genes in tumor-adjacent normal tissue, but no in tumor tissue in each pathway was associated with recurrence (Table 17).

TABLE 17
Local recurrence in rectal cancer and gene mRNA levels by pathway in tumor and
tumor-adjacent tissue

| | Tumor-adjacent | | | | |
|---|---|---|---|---|---|
| | normal tissue | | | Tumor tissue | |
| | Recurrence pattern | | | Recurrence pattern | |
| Pathway | Yes | No | Pathway | Yes | No |
| 5-FU metabolism | | | | | |
| TS ≦ 1.9 and DPD ≦ 1.5 | 21 (72%) | 8 (28%) | TS ≦ 2.8 and DPD ≦ 0.6 | 14 (54%) | 12 (46%) |
| TS > 1.9 or DPD > 1.5 | 3 (18%) | 14 (82%) | TS > 2.8 or DPD > 0.6 | 11 (39%) | 5 (61%) |
| P value^{b} | 0.003 | | | 0.66 | |
| DNA repair | | | | | |
| ERCC1 ≦ 2.2 and | 20 (69%) | 9 (31%) | ERCC1 ≦ 0.7 and | 1 (11%) | 8 (89%) |
| RAD51 ≦ 1.4 | | | RAD51 ≦ 2.7 | | |
| ERCC1 > 2.2 or | 4 (24%) | 13 (76%) | ERCC1 > 0.7 or | 23 (53%) | 20 (47%) |
| RAD51 > 1.4 | | | RAD51 > 2.7 | | |
| P value^{b} | 0.038 | | | 0.17 | |

^{b}Based on the Fisher's exact test, but after 2,000 bootstrap-like simulations to adjust for selection of optimal cut point for recurrence

Recursive Partitioning (RP) Analysis of Recurrence

The mRNA levels of 6 genes in tumor tissue and in tumor-adjacent normal tissue as well as sex and ethnicity were considered in the RP analysis (a total of 14 predictors). The expression levels of EGFR and VEGF in tumor-adjacent normal tissue and RAD 51 in tumor tissue were chosen as splits to classify patients in terms of recurrence probability. Four terminal nodes were identified based upon mRNA levels of these three genes. The high risk group for recurrence included Group 2 and Group 4. The low probability group for recurrence included Group 1 and Group 3.

Discussion

This study was designed in order to identify a gene expression profile that may be associated with the likelihood of recurrence in patients with locally advanced rectal cancer treated with adjuvant chemoradiation therapy. Candidate genes were selected that had previously been shown to be involved in the metabolism of 5-FU (TS, DPD), in DNA repair (ERCC-1, RAD51), and in angiogenesis and radiation sensitivity (VEGF, EGFR).

In the intratumoral gene expression analysis, elevated VEGF mRNA levels were associated with recurrence. VEGF is a potent angiogenic factor, and its expression has been shown to be correlated with microvessel count and metastasis. Various studies have consistently shown intra-tumoral VEGF expression levels correlate with poor clinical outcome in an array of neoplasms including bladder, esophageal, gastric, and colon. In the same way, this data reflects the aggressive nature of VEGF-high expression tumors which lead to worse clinical outcome. High expression levels of VEGF have been associated with resistance to radiation therapy, since hypoxia is one of the major mechanism of radiation resistance.

Interestingly, no significant associations between intra-tumoral gene expression levels of the other candidate genes (TS, DPD, ERCC-1, Rad51, and EGFR) and tumor recurrence were found. In this gene expression analysis of tumor-adjacent normal rectal tissue significant (TS, DPD, VEGF) and marginal associations (DPD, ERCC-1) between recurrence and the mRNA levels of five of the six investigated genes were found. As expected, elevated levels of TS and DPD were associated with recurrence. TS is the rate-limiting enzyme needed for the methylation of dUMP to dTMP, thus being essential for DNA synthesis. The active metabolite of 5-FU binds to TS which leads to its inactivation. DPD is the first, and rate-limiting enzyme that commences the catabolism of TS. Numerous studies, with a few exceptions, have shown the predictive potential of intra-tumoral TS and DPD gene expression with fluoropyrimidine therapy in both locally advanced or metastatic gastric and colorectal cancer.

The ERCC1-XPF complex is involved in two distinct DNA-repair pathways: the nucleotide excision repair pathway (for intra-strand DNA repair) and the recombination-dependent removal of DNA inter-strand cross-links. The latter pathway may play a role in radiation sensitivity, especially under condition of hypoxia. In this study, high levels of ERCC-1 gene expression correlated with a higher risk of recurrence probably due to increased ability to repair ionizing radiation-induced DNA damage.

Higher expression of genes involved in angiogenesis and radioresistance in tumor-adjacent normal tissue were also associated with a significantly increased chance for treatment failure. In fact, exploratory tree analysis of 14 predictors (which included gene expression levels of tumor and tumor-adjacent normal tissue) yielded tumor-adjacent normal tissue EGFR and VEGF mRNA levels as the strongest predictors. Interestingly, intra-tumoral VEGF was not chosen as a split. In vitro studies have shown that VEGF overexpression can protect tumor cells from the cytotoxic effects of ionizing radiation. Several studies have linked EGFR overexpression to radioresistance in a variety of neoplasms including rectal cancer.

The reason for which tumor-adjacent normal tissue gene expression of key candidate genes was a better overall predictor for early recurrence compared to intratumoral gene expression is unclear. One possible explanation is that the gene expression analysis of these candidate genes in the adjacent normal tissue may reflect the regulation of the microenvironmental milieu. Interestingly the genes associated with recurrence are in the metabolic pathway of 5-FU, DNA repair and angiogenesis. Whether these genes directly are responsible to the treatment failure or if they reflect the tumor profile is unknown. Surprisingly VEGF expression in the tumor and adjacent normal tissue has been shown to be associated with recurrence even the cut off levels were different, supporting again the idea that the genetic make up in the adjacent normal tissue plays a critical role for tumor recurrence. Local recurrences usually come from tumor cells left behind in the normal tissues or lymph nodes. These cells do underlie the microenvironmental pressure as well as the immunopresponse. VEGF for example is critical for the maturation of dendritic cell and their migration and expression of VEGF in tumor associated macrophages have been shown to be a good prognostic marker in stage III colon cancer. The molecular characteristics including gene expression may be partly regulated by the tissue specific factors regulating the integrity of the environment. Without being bound by any theories, the Applicants speculate that the pressures regulating the gene expression levels of “normal” tissues may in part influence the expression pattern of the residual tumor cells, which may allow them to survive. However, tumor cells with mutations in critical pathways such as p53 or kras may be able to survive this microenvironment due to their own stimulated growth pathways which are resistant to the influences of the microenvironment.

Microenvironment refers to the milieu surrounding a cluster of cells (tumor among normal or vice versa) where cell-adhesion, angiogenesis, apoptosis, and growth factor regulators, as well as physiologic pressures such as hypoxia exert their influence in a complex manner.

Previous studies investigating the biologic mechanisms of metastasis formations have shown that the microenvironment of the target organs has influence on tumor growth and chemo-sensitivity. For example, paracrine EGFR stimulation by stroma cells activated the tumor growth in human bladder cancer cell lines. The organ environment may influence the tumor cell functions, lead to a production of degradative enzymes and modulate the gene expression of tumor cells, resulting for example in an overexpression of EGFR mRNA.

Similar to the mechanisms involved in the formation of organ metastases, local microenvironment could influence the function of tumor cells by regulating gene expression levels which may be associated with local tumor recurrence. In vitro experiments have shown previously that cells react to radiation treatment regardless whether they were treated with radiation themselves or whether they were just neighboring the treated cells. Modulations of bystander cells have been shown on DNA (sister chromatid exchanges) and RNA levels (modulated expression levels of p53, p21, and MDM2). The signals leading to these changes were transmitted from irradiated to bystander cells by gap junction mediated intercellular communication. As a molecule involved in these contacts, IL-8 has been demonstrated to play an important role in cell-to-cell communications.

Previous studies have suggested that the organ environment, hosting the tumor, has profound effects on the response of tumor cells to the treatment with drugs. In vivo studies revealed a dependence of the response to 5-FU, doxorubicin, and adriamycin treatment on the site of implantation of the tumors in nude mice. Differences in the distribution of the drug can be excluded as a possible reason for the diverse response. Furthermore, Dalton et al. suggested that extracellular effectors such as cytokines or matrix components might play an important role in the emergence of drug resistance. He demonstrated that stromal tissue secrets IL-6 and expresses fibronectin that directly correlates with sensitivity to chemotherapy.

In a recent report, gene expression levels of folate metabolism enzymes in normal-appearing colonic mucosa adjacent to tumor, but not in tumor tissue, were associated with survival in colorectal cancer patients. The authors suggested that gene expression from normal-appearing tissue (obtained at least 10 cm from tumor) may be more consistently reliable due to the homogeneity of the sample, in contrast to the heterogeneous nature of samples obtained from tumor biopsies. They also suggested that normal-appearing “transitional” mucosa exhibits altered gene expression by adjacent tumor, thus predicting tumor-specific survival. The cohort was a heterogeneous sample of colon and rectal cancer patients at Duke's stages A to D with unspecified therapy. The study-points out to a potentially significant role of determining “normal” tissue gene expression as marker of clinical outcome, and underscores the importance of better understanding the true nature of normal-appearing mucosa, either adjacent or distant from carcinoma.

These data demonstrate for the first time that the genetic profile of the tumor-adjacent normal tissue was more likely to be associated with early treatment failure than intratumoral gene expression.

### Example 6

### CPT-11 Specific Gene Polymorphisms Predict Clinical Outcome in Metastatic Colorectal Cancer Patients

Irinotecan (CPT-11), a topoisomerase I inhibitor, is approved for the use of both first- and second-line chemotherapy in metastatic colorectal cancer (CRC) patients. As of yet, no reliable prognostic factors have been identified for predicting the clinical outcome of CPT-11 treatment. Specific gene polymorphisms that are known to be involved in general drug metabolism, specifically the Irinotecan metabolic pathway were identified, which included members of the ATP-binding cassette transporter subfamily (ABCB1 C1236T, C3435T, and G2677A, ABCG2 T623C, and ABCC2 3972), carboxylesterase 1 (CES1 A1525C), carboxylesterase 2 (CES2 G-140C), hepatic organic anion transport protein (OATP-C A388G and T521C) and cytochrome P450 (CYP3A4).

Patients

Fifty-four patients with metastatic colorectal cancer (UICC stage IV) who were treated with first-line 5-FU/Leucovorin and Irinotecan chemotherapy at the University of Southern California/Norris Comprehensive Cancer Center, Los Angeles between 1999 and 2003 were eligible for the present study. A portion of these 54 patients was enrolled in the 3C-00-4 (12 patients) and 3C-01-4 (19 patients) clinical trials. The remaining 23 patients were not included in a clinical trial, though they were all treated at the University of Southern California/Norris Comprehensive Cancer Center, Los Angeles. This study was investigated at the USC/Norris Comprehensive Cancer Center and was approved by the Institutional Review Board of the University of Southern California for Medical Sciences. Patient data was collected retrospectively, and all patients involved in the study signed informed consents.

Those patients classified as responders (R) to therapy demonstrated a decrease in tumor burden by 50% or more for at least six weeks. CT imaging for response was performed every six weeks. Patients with evaluable but non-measurable disease were classified as demonstrating complete response (CR) only if the tumor and all evidence of disease had disappeared. Progressive disease (PD) was defined as a 25% or more increase in tumor burden (compared to the smallest measurement) or the appearance of new lesions. Patients, who did not experience a response and did not progress within the first 12 weeks following the start of CPT-11/5-FU -based chemotherapy, were classified as having stable disease (SD).

Toxicity

All toxicity measurements were graded in accordance with the National Cancer Institute Common Toxicity Criteria 1.0 (Apr. 16, 2003).

Polymorphism Investigation

Blood samples were collected from each patient, and genomic DNA was extracted using the QiaAmp kit (Qiagen, Valencia, Calif.). Polymorphisms for each patient were determined using the PCR-RFLP assay. The alleles were separated on 4% Nusieve ethidium bromide-stained agrose gel.

Results

This study cohort was comprised of 31 men (57%) and 23 women (43%) with a median age of 56 years (range: 34-77 years). Participants represented four ethnicities: 29 Caucasian (54%), 12 Asian (22%), 10 Hispanic (19%), and three African-American (6%). Three patients (6%) demonstrated complete response, 20 patients (38%) showed partial response, 24 patients (45%) continued with stable disease, and six patients (11%) were found to have progressive disease. One patient was invaluable for response data. Of the 54 patients in the study, 25 (47%) experienced Grade 0-2 toxicity, while 28 (53%) experienced Grade 34 toxicity. One patient was inevaluable for toxicity data.

ABCB1 Gene Polymorphisms

Fifty-four (54) patients for the ABCB1 C1236T polymorphism; the assay was successful for 47 patients. Thirty-six percent (17/47) of these patients were found to have the homozygous CC genotype, 45 percent (21/47) the heterozygous CT genotype, and 19 percent (9/47) the homozygous TT genotype. A significant correlation trend (logrank test; P=0.06) was detected between the C1236T polymorphism and time-to-tumor progression. Moreover, combined analysis detected a statistically significant difference in time-to-tumor progression between patients carrying the C allele (CT heterozygous or CC homozygous) and patients not carrying the C allele (homozygous TT) (logrank test; P=0.01); patients positive for the C allele demonstrated the longer time-to-progression.

Forty-nine (49) of the eligible 54 patients were evaluated for the ABCB1 A2677G polymorphism. Twenty-seven percent (13/49) of the patients were found to have the homozygous AA genotype, twenty-four percent (12/49) the homozygous GG genotype, and forty-nine percent (24/49) the heterozygous AG genotype. Additionally, the study successfully assessed the ABCB1 C3435T polymorphism for 50 of the 54 eligible patients. Twenty-eight percent (14/50) of the patients were found to have the homozygous CC genotype, 14 percent (7/50) the homozygous TT genotype, and 58 percent (29/50) the heterozygous CT genotype. Though Hoffmeyer et al. found a correlation between the C3435T polymorphism and overexpression of the P-glycoprotein, this study did not find any statistically significant clinical data conferring their finding However, it was found that both the A2677G and C3435T polymorphisms demonstrated a significant association with time-to-tumor progression (logrank test; P=0.06 and P=0.15 respectively). In both cases the variant alleles (G and T) demonstrated poorer prognosis than did the wild type alleles (A and C).

OATP-C Polymorphisms

There was no significant data implicated in the polymorphisms of CES1, CES2, ABCC2, ABCG2, and CYP3A This study detected no statistically significant association between the OATP-C A388G polymorphism and clinical outcome. Fifty out of the eligible 54 patients were successfully evaluated for the OATP-C T521C polymorphism. Seventy-two percent (36/50) of these patients were found to have the homozygous TT genotype, 18 percent (9/50) the homozygous CC genotype, and 12 percent (6/50) the heterozygous CT genotype. It was found for this OATP-C T521C polymorphism that patients homozygous for the variant C allele showed a statistically significant (logrank test; P=0.028) greater incidence of Grade 34 toxicity than those patients with the other two possible genotypes.

Discussion

This study was designed to search for the first reliable molecular predictive markers for clinical outcome in CRC patients treated with first-line CPT-11 based chemotherapy. The goal was to investigate common polymorphisms in genes involved in CPT-11 metabolism and in general and specific drug influx/efflux pathways. Unfortunately, the study found no significant correlation or association between the CES1, CES2, ABCC2, ABCG2 and CYP3A4 polymorphisms and clinical outcome. It did, however, detect a significant relationship between both the ABCB1 and OATP-C gene polymorphisms and clinical outcome.

### Example 7

### Association of Genomic Profiling with Pelvic Recurrences in Patients with Rectal Cancer Treated with Chemoradiation

Patients

The analyses of the present study were performed based on results from 92 eligible patients diagnosed with either stage II or III rectal cancer. This study was investigated at the Norris Comprehensive Cancer Center and approved by the Institutional Review Board (IRB) of the University of Southern California for Medical Sciences. A tumor was considered to be a rectal cancer if a portion of the tumor was situated below the peritoneal reflection or if the lower margin of the tumor was within 12 cm of the anal verge on endoscopy (Tepper (2002) J. Clin. Oncol. 20:1744). Out of 92 patients, seventy-three patients were treated with adjuvant infusional 5-FU chemotherapy combined with pelvic radiation. Nineteen patients were treated with neo-adjuvant chemoradiation therapy. Forty-one percent (38/92) of patients developed local tumor recurrence during the follow-up time. The age, ethnicity, and follow-up information for each subject were obtained from the retrospective chart reviews, and all patients involved in the study signed informed consent.

Of the 92 subjects in this study, 60 were treated exclusively at the University of Southern California Norris Cancer Center or University of Southern California/Los Angeles County Hospital. 15 of these 60 patients (22%) experienced local recurrence (University of Southern California Norris Cancer Center—15%; University of Southern California/Los Angeles County Hospital—37%). The remaining 32 patients were originally treated at an outside facility and referred to USC/Norris for treatment of recurrent disease or for check-up.

Genotyping

A tissue sample was collected from each patient and genomic DNA was extracted from paraffin-embedded tissue using the QiaAmp kit (Qiagen, Valencia, Calif.). All samples were analyzed using a PCR-RFLP-based technique. The PCR reaction volume was 50 μL. After restriction enzyme digestion, the resulting PCR fragments were visualized in 3-4% agarose gel. Primer sequences, restriction enzymes, and references for the genotype analyses are known in the art.

Statistical Analysis

In this analysis, recurrence status was categorized into two groups: (1) having recurrence within 5 years of completion of adjuvant chemoradiation; (2) being recurrence-free within 5 years of completion of adjuvant chemoradiation. The associations of recurrence with patient characteristics including demographic (age, sex, and race), pretreatment information (grade, T-stage, N-stage, and type of surgery), and type of therapy (neoadjuvant vs. adjuvant therapy) were summarized using a contingency table and were formally tested by Fisher's exact tests.

The associations between each polymorphism variable and recurrence as well as baseline characteristics were evaluated by the Fisher's exact test and summarized using contingency tables.

Finally, a classification and regression tree (CART) method based on recursive partitioning (RP) was used to explore gene polymorphisms for identifying homogenous subgroups for recurrence after completion of chemoradiation. The RP analysis is a nonparametric statistical method for modeling a response variable and multiple predictors. The PR analysis includes two essential processes: tree-growing and tree pruning. The tree-growing procedure starts with all patients in one group and makes a series of binary splits based on predictors that defined the subgroups most distinct in tumor recurrence. At each partitioning, the tree-growing method examines all possible splits for all gene polymorphism variables and baseline variables to select the best cut point. In case of missingness in the primary splitting gene polymorphism variable, the patients are classified based on alternative splits (surrogates). The process is repeated until the terminal nodes reach a minimum size (n=5). The splitting rule of RP is based on the Gini diversity index (1—the sum of squared probabilities over all levels of response). After the tree-growing procedure has completed, the tree-pruning process starts to produce a sequence of simpler subtrees through assessing the misclassification error associated with a particular subtree. The goal of tree-pruning is to select a final tree from the set of subtrees that minimize both the relative cost, a measure of the misclassification error, and the number of terminal nodes. The RP analysis included all patients with any gene polymorphism variables available (n=90).

All reported P values were two-sided. All analyses were performed using the SAS statistical package version 8.2 (SAS Institute Inc. S. SAS/STAT® User's Guide, Version 8. Cary, N.C.: SAS Institute Inc., 1999), and CART 5.0 (Steinberg D, and Colla P. CART: Tree-Structured Non-Parametric Data Analysis. San Diego, Calif.: Salford Systems, 1995).

Results

Paraffin-embedded tissue samples from 90 patients were obtained for the genotype analysis. This group of 90 patients included 34 (38%) women and 56 (62%) men. The median age was 53 years (range 26-80 years). The ethnic backgrounds were as follows: 68% (61/90) Caucasian and 32% (29/90) others. Neoadjuvant (preoperative) radiotherapy was given to 23 patients and postoperative adjuvant radiotherapy to 67 patients. Twenty-five percent (23/90) had T2 and 75% (67/90) of the study participants had T3 tumor stages. In addition, 82% (74/90) of the patients had grade I/II tumors and 18% (16/90) had grade III. Furthermore, 40% (36/90) had node-negative, 50% (45/90) had node-positive status, and 10% (9/90) were inevaluable for node status due to transanal resection. Fifty-one percent (46/90) of the participants had pelvic recurrences. 11 (12%) patients developed distant metastases but did not have local recurrence. Twelve percent (11/92) of the study participants died from the disease during the follow-up period.

Risk of Recurrence Analysis-Clinical Characteristics

Of the 92 participants for this study, the median follow-up period was 51.1 months (range: 1.1-143.1 months) and the median time to tumor recurrence for all patients was 57.0 months (95% Cl, 40.5-143.1+months). The age, gender, and ethnicity were not associated with time to tumor recurrence. A trend for association between node status and pelvic recurrence (p=0.09), but there was no significant correlation between the tumor grade, T-stage, or surgery type and time to local failure.

Of the eligible study participants, results from the assays for the aforementioned polymorphisms could be obtained as follows: TGFβ: 89 patients; VEFG, p53 codon72: 88 patients; TS 5′UTR, GSTM1, GSTP1-105, APE1, RAD51, MMP3, COX-2, ICAM-1, p53-13964, CCND: 87 patients; TS5′SNP, TS3′UTR, GSTT1, XRCC3, FGFR4: 86 patients; IL-8: 77 patients.

Association Between the Germ-Line Polymorphisms of p53 (₁₃₉₆₄^{GC}), ERCC1-118, XRCC3 genes and clinical characteristics.

Evaluation of these polymorphisms of interest revealed the following association with demographic and clinical variables: ERCC1 -118 and race (p<0.001); XRCC3 and race (p=0.012); APE1 and node status (p=0.041); p53 (₁₃₉₆₄^{GC}) and node status (p=0.006); p53 (₁₃₉₆₄^{GC}) and grade (p=0.043).

Univariate Analysis of Germ-Line Polymorphisms and Time to Tumor Recurrence

The IL-8 polymorphism was significantly correlated with risk of recurrence among the study participants (Table 19). Assessment of the polymorphisms of Cox-2, GSTP-1, TGF-β, and p53 indicated a trend for association with risk of recurrence. Analyses of the remaining polymorphisms failed to individually show a significant association with pelvic recurrence.

IL-8. Thirty-two patients (42%.) were homozygous for the T allele, thirty-seven patients (48%) were heterozygous ANT, and 8 patients (10%) were homozygous for the A allele. Patients with A/A genotype were at increased risk for local recurrence in 5 years (p=0.029). Thirty-eight percent of patients carrying T/T genotype experienced recurrence (12/32), 57% of patients who were heterozygous experienced recurrence (21/37), and 88% of patients homozygous for the A allele experienced local recurrence (7/8).

Cox-2. The Cox-2 genotype distribution was as follows: 84% (73/87) had G/G, 16% (14/87) had G/C, and 0% (0/89) had C/C genotypes. Fifty-six percent (41/73) of the patients with homozygous G allele and 29% (4/14) of those with heterozygous genotype showed evidence of pelvic recurrence, respectively. Therefore, possession of C allele that results in lower Cox-2 expression was associated with decreased risk for local failure when compared to having the G/G genotype (p=0.081).

GSTP1-105. Thirty-eight patients (44%) possessed the homozygous ¹⁰⁵lle/¹⁰⁵lle GSTP1 genotype, 6 patients (7%) had homozygous ¹⁰⁵Val/¹⁰⁵Val genotype, and 43 patients (49%) were heterozygous. Pelvic recurrence was observed in 83% (5/6) of the patients with ¹⁰⁵Val/¹⁰⁵Val genotype, while 58% (22/38) of those with ¹⁰⁵lle/¹⁰⁵lle genotype and 42% (18/43) of those heterozygous showed evidence of local failure. Patients with ¹⁰5Val/¹⁰⁵Val genotype were at increased risk for tumor recurrence (p=0.089).

TGF-β. Thirty-five out of 89 (39%) patients were homozygous T/T. Forty patients were heterozygous C/T (45%), and 14 were homozygous C/C (16%). Local recurrence occurred in 13 of 36 TfT carriers (37%), 24/40 CfT carriers (60%), and 8 of 14 C/C carriers (57%). Therefore, patients carrying the C allele experienced more recurrence (p=0.12).

Recursive Partitioning (RP) Analysis of Recurrence

The 20 genomic polymorphism variables as well as lymph node status were considered in the RP analysis (a total of 21 predictors). The first split was based on lymph node status, with the best cutoff N0 or N1 versus N2. For patients with N2, no further subgroups could be identified. Among those with N0 or N1 the next division was according to the IL-8 genotype. For patients with A/A or A/T of IL-8 further splits were made with ICAM-1, TGF-,6, and FGFR4 polymorphisms. The polymorphisms of 4 genes involved in tumor microenvironment in addition to lymph node status were chosen as splits to classify patients in terms of recurrence probability. Six terminal nodes were fit. The high risk group for recurrence included Group 1, Group 2, and Group 6. The low probability group for recurrence included Group 3, Group 4, and Group 5.

Discussion

While adjuvant chemotherapy and radiation lead to a noticeable improvement in local control among those with rectal carcinoma, the choice of optimal therapy may be compromised by a wide inter-patient variability of treatment response and host toxicity. Since the rate of inactivation of the administered drug compound may establish its effectiveness in the tumor tissue, the purpose of this study was to evaluate the influence of genomic variations on different cellular mechanisms that may modify therapy efficacy. Germ-line polymorphisms in the genes of IL-8, COX-2, GSTP1, and TGF-β that may be individually associated with both radiation efficacy and tumor recurrence in patients with rectal cancer treated with chemoradiation were identified. In addition to univariate analysis, a more comprehensive approach using CART analysis was taken. Using CART analysis, patient risk of recurrence based on clinical factors as well as gene polymorphism was stratisfied. Patients presenting with N2 disease as well as unfavorable polymorphisms in the IL-8, ICAM-1, TGFβ, and FGFR4 genes experienced significantly greater recurrence than patients without this profile.

Univariate Analysis

The angiogenic response in the microvasculature is associated with changes in cellular interactions between adjacent endothelial cells (ECs), pericytes, and surrounding ECM (Gupta (2003) World J. Gastro. 9(6):1144). Clearly, a gamut of evidence suggests the importance of angiogenic factors and cell adhesion molecules in cancer progression. IL-8, Cox-2, ICAM-1, TGF-β, and FGFR4 are major regulators of angiogenesis and cell adhesion.

Increased IL-8 expression has been associated with angiogenesis, advanced disease state, lymph node metastasis, shortened survival, and recurrence in non-small-cell lung cancer (Yuan 2000), supra. Also, an increase in serum IL-8 levels has been associated with colorectal cancer patients, specifically patients with lung or liver metastases (Ueda (1994) J. Gastro. 29:423). IL-8 polymorphism was found to be significantly associated with risk of recurrence in both univariate analysis and in the regression-tree analysis. Patients carrying the A variant allele, which has been associated with increased IL-8 expression in vitro (Hull (2000) Thorax 55:1023), experienced more recurrence than those patients carrying homozygous T allele.

An in vitro study using cell lines from a murine sarcoma revealed that pretreatment of these cells with SC-236, a selective Cox-2 inhibitor, dramatically improved tumor cell radiosensitivity (Raju (2002) Int. J. Rad. Oncol. Biol. Phys. 54:886), implying that patients who are genetically predisposed to producing higher levels of Cox-2 may respond poorly to radiation therapy, and thus be at an increased risk for local failure. In another study, however, Cox-2 expression lacked significance as a prognostic factor for local control and survival in patients with rectal carcinoma, although these results have indicated that Cox-2 may be correlated with an increased risk of hematogenous metastatic spread (Petersen (2002) Anticancer Res. 22:1255). AG→C SNP at codon 765, which has been associated with altered expression levels of COX-2 reporter gene (Papafili (2002) Artherosclerosis 22:1631) was examined. The resulting data suggest that patients with genotypes leading to decreased Cox-2 expression may be less prone to pelvic recurrence. However no patients in this population carried the homozygous variant allele, and the results did not reach statistical significance, therefore caution in interpreting this data and a larger population to validate these results are necessary.

In the GST super-family, polymorphisms in the GSTT1, GSTM1, and GSTP1 genes were examined because it was hypothesized that patients with genotypes leading to lower GSTP1 activity may be more responsive to chemoradiation, leading to lower incidence of local failure. Surprisingly, GSTP1 ¹⁰⁵lle allele, leading to higher enzymatic activity, was associated with low pelvic recurrence while the ¹⁰⁵Val allele was associated with high recurrence. Interestingly, overexpression of GSTP1 has been implicated in preventing local relapse in patients with breast and cervical cancer treated with radiation therapy (Silverstrini (1997) J. Natl. Cancer Inst. 89:639 and Daidone (1997) Int. J. Oncol. 10:41). Moreover, deactivation of GST was associated with poor response to radiotherapy in patients with squamous cell carcinoma of buccal mucosa, and suggested that low GST may lead to accumulation of reduced glutathione, an important cellular antioxidant and a scavenger of free radicals produced during radiotherapy (Rawal (2001) Int. J. Rad. Biol.). Unlike in the chemotherapy setting where GSTP1 plays a role in cellular detoxification, and without being bound by any theory, this study suggests that lower GSTP1 leads to higher glutathione that quenches the radiation-induced free radicals, consequently limiting the efficacy of radiotherapy in patients with rectal cancer. However, based on the opposing effects of the GSTP1-105 polymorphism on chemotherapy and radiation, the predictive value of this polymorphism in patients with rectal cancer requires further evaluation. The deletion polymorphisms of GSTM1 and GSTT1, which are associated with abolished enzyme activity (London (2000) Lancet 356:724), were not associated with time to pelvic recurrence. Predominant expression of the GSTP1 subclass compared to GSTM1 and GSTT1 in colorectal epithelial and tumor tissue may in part explain this phenomenon (Moscow (1989) Mol. Pharm. 36:22).

CART Analysis

In addition to univariate analysis of polymorphisms and clinical data, a decision algorithm was developed to screen for risk of recurrence using CART analysis. Eighty-five percent (85%) of patients with N2 disease experienced recurrence, making lymph node status the best stand-alone predictor of recurrence. Among patients with N0 or N1 disease, those carrying the T/T allele had a better prognosis. Patients carrying A allele were further distinguished by ICAM-1 polymorphism, with patients carrying T/T allele in a poor prognosis group. Those patients carrying C allele in ICAM-1 gene were further distinguished by TGF-β polymorphism. Patients carrying homozygous T allele in TGF-β were placed in a good prognosis group, while patients carrying C allele were further subdivided again by FGFR4. Patients who were heterozygous for the FGFR4 polymorphism had a high risk of recurrence, while patients homozygous for the wild-type or variant allele were in a low risk group. Thus, CART analysis identified three high-risk groups and three low-risk groups for recurrence of disease.

The ability to seek out patients who are at increased risk for experiencing tumor recurrence and/or those who may be more susceptible to clinical toxicity will significantly impact the development of more effective but less toxic therapy regimens in the future. These findings may contribute to identifying categories of high-risk patients and developing tailored treatment strategies. For patients with rectal cancer who may be more prone to experiencing higher frequency of tumor recurrence due to their genetic predisposition, more specific and aggressive chemoradiation therapy may be necessary.

### Example 8

### Genomic Profiling as Predictor of Gastrointestinal (GI) and Neurological Toxicity in Patients with Advanced Colorectal Cancer Treated with Platinum-Based Chemotherapy

A significant association was shown between functional genomic polymorphisms in genes involved in drug metabolism and DNA-repair and outcome to platinum-based chemotherapy in advanced colorectal cancer. This experiment reports an association between relevant genomic polymorphisms and toxicity to platinum-based chemotherapy.

Applicants show herein that polymorphism in a gene selected from the group consisting of XPD, GSTP1, TS, and COX2 promoter predicts GI or neuro-toxicity to 5-FU/oxaliplatin chemotherapy.

Patients

As of December 2003, 168 patients with advanced refractory colorectal cancer have been enrolled in this study. Patients received 5-FU (200 mg/mz/day) as continuous infusion and oxaliplatin (130 mg/mz), in three week cycles. The first 130 patients with sufficient follow-up time were included in the analysis. Polymorphisms in genes involved in drug metabolism, DNA-repair, sodium channel, angiogenesis, and drug targets (XPD, TS, ERCC1, GST, COX2, R19K sodium channel) were determined and their potential associations with gastrointestinal (GI) and neurological toxicities assessed.

Results

The group of 130 patients comprised 68 males and 62 females, their median age being 60 yrs (25-87). The median number of cycles received was 4. Sixty-two percent (72/123) of patients experienced G3+overall toxicity, 42% (52/123) GI toxicity, and 9% (11/123) neurotoxicity. Female patients were at a higher risk for experiencing G3+overall and GI toxicity (RR=2.07 and 2.41, log-rank p<0.001). COX2 promoter C-allele and GSTP 1 Val 05 Val polymorphism were associated with mucositis (G1 +)(RR=1.73, p0.049; RR=3.88, p<0.001). A trend for neurotoxicity (G2+) was seen for TS 5′ GC (SNP) in patients homozygous for the C-allele (RR=2.35, p=0.08). After stratification by gender, significant associations between the polymorphisms XPD Gln75I Gln and G3+overall (RR2.19; p0.017) and GI toxicity (RR=2.65; p=0.019) were found.

Genomic polymorphisms in XPD, GSTP1, TS, and COX2 promoter predicts GI or neuro-toxicity to 5-FU/oxaliplatin chemotherapy.

### Example 9

### MnSOD and GPx-1 Polymorphisms in Relation to Local Recurrence in Patients with Rectal Cancer Treated with Chemoradiation

Manganese superoxide dismutase (MnSOD) and Glutathione peroxidase-1 (GPx-1) are two enzymes that scavenge ROS (reactive oxygen species) such as superoxide and hydrogen peroxide and protect cells from oxidative damage. MnSOD reduces superoxide to oxygen and hydrogen peroxide, and GPx-1 reduces hydrogen peroxide to water. Radiation therapy produces an excess of ROS, which results in DNA damage, cellular destruction, and tumor degradation. In vitro and in vivo studies show that increasing the levels of MnSOD and GPx-1 lowers the level of ROS in the cell.

Two functional polymorphisms, an Ala-9Val SNP in the mitochondrial targeting sequence of the MnSOD gene and a Pro/Leu SNP at codon 197 near the C-terminus of the GPx-1 protein were tested for correlation with local recurrence in chemoradiation-treated rectal cancer patients to determine if the activity levels of MnSOD and GPx-1 may-be directly related to the efficacy of chemoradiation treatment and hence may influence risk of local recurrence.

Methods

DNA was extracted from rectal tissue and blood from 92 patients with locally advanced rectal cancer treated with neoadjuvant or adjuvant chemoradiation, of which 38 had local recurrence and 54 had no recurrence. MnSOD Ala-9Val polymorphism and GPx-I Pro-198Leu polymorphism were tested using PCR-RFLP method.

Results

A combined analysis of the genotypes showed patients carrying at least one unfavorable genotype (homozygous Ala for MnSOD and homozygous Leu for GPx-I). had a greater risk of recurrence (relative risk: 2.36, 95% Cl) when compared to patients possessing no unfavorable genotypes (p=0.01). Individually, MnSOD and GPx-1 showed trends for tumor recurrence (p=0.087 for GPx-1 and p=0.13 for MnSOD).

The data show that polymorphisms in free radical scavengers MnSOD and GPx-1 are potential prognostic markers for local recurrence in rectal cancer and can predict chemoradiation efficacy. Thus, this invention provides a method for selecting a therapeutic regimen for treating rectal cancer in a patient, the method comprising screening a suitable cell or tissue sample isolated from the patient for one of these polymorphisms. The presence of one of these polymorphisms would be predictive of the likelihood of future lymph node involvement. A therapeutic regimen to combat this likelihood should be considered for this patient.

### Example 10

### Angiogenic Profiling Predicts Site of Metastasis in Patients with Colorectal Cancer

Cancer metastasis is a highly complex process that involves aberrations in gene expression leading to transformation, growth, angiogenesis, invasion, dissemination, survival in the circulation, and subsequent attachment and growth in the organ of metastasis. Angiogenesis facilitates metastasis formation and changes of tumor cell-extracellular matrix interactions at the metastatic site. The establishment of metastatic lesions depends on the activation of multiple angiogenic pathways. Factors involved in the angiogenesis of liver metastasis have been identified: vascular endothelial growth factor (VEGF), interleukin-8 (IL-8), and platelet-derived endothelial cell growth factor. Functional polymorphisms of genes implicated in the angiogenic pathways were hypothesized to predict distant metastasis in patients with colorectal cancer. Sixty (60) out of 638 patients with colorectal cancer treated at USC during 1998 to 2000 were identified. 10 had only peritoneal carcinomatosis with a median follow up of 17 (10.6-25.3) months; 50 patients presented with liver and or lung metastasis with a median follow up of 34.3 (11.6-61.3) months. Ten different gene polymorphisms of angiogenic factors (VEGF +936 C/T, IL-8 -251 A/T, CXCR1 +2607 Ser/Thr, IL-10 -1082 G/A, TGF-β+869 Leu/Pro, IL-6 -174 G/C), proteinases (MMP-I -1607 1G/2G, MMP-3 -1171 5A/6A, MMP-9 -1562 CIT) and adhesion molecule (E-Cadherin -160 C/A) were examined using PCR-RFLP. TGF-β +869 Leu/Pro and MMP-1 -1607 1 G/2G are significant different between local and distance metastatic site. These data show that angiogenic profiling predicts distant metastases in patients with colorectal cancer.

Thus, this invention provides a method for selecting a therapeutic regimen for treating colorectal cancer in a patient, the method comprising screening a suitable cell or tissue sample isolated from the patient for TGF-β +869 Leu/Pro or MMP-1 -1607 1 G/2G polymorphism. The presence of one of these polymorphisms would be predictive of the likelihood of metastasis in colorectal cancer patients. A therapeutic regimen to combat this likelihood should be considered for these patients.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

We claim:

1. A method for selecting a therapeutic regimen for treating a cancer in a patient, the method comprising screening a suitable cell or tissue sample isolated from said patient for a genomic polymorphism or genotype that is correlative to treatment outcome of the cancer.

2. The method of claim 1, wherein the cancer is treatable by the administration of a chemotherapeutic drug or agent selected from the group consisting of fluoropyrimidine, a platinum drug, a topoisomerase inhibitor and an anti-EGFR antibody or small molecule.

3. The method of claim 1, wherein the cancer is selected from the group consisting of colon cancer, rectal cancer, metastatic colorectal cancer and non-small cell lung cancer.

4. The method of claims 1 or 2, wherein the cancer treatment further comprises radiation therapy.

5. The method of claim 1, wherein the therapeutic regimen comprises the administration of 5-fluorouracil (5-FU).

6. The method of claim 1, wherein the therapeutic regimen comprises the co-administration of 5-FU and oxaliplatin.

7. The method of claim 2, wherein the anti-EGFR antibody comprises an active fragment or variant of cetuximab antibody.

8. A method to identify a putative therapeutic target comprising detecting a mutation or a polymorphism that alters stability of mRNA, thereby identifying a putative therapeutic target.

9. The method of claim 8, wherein the mRNA is transcribed from a gene coding a mRNA binding protein.

10. The method of claim 9, wherein the mRNA binds to a mRNA binding protein.

11. The method of claim 9, wherein the mRNA is encoded by a gene encoding a product involved in drug metabolism.

12. A method to identify a putative therapeutic target comprising detecting a mutation or a polymorphism that affects drug metabolism or toxicity.

13. The method of claim 12, wherein the gene encoding a product involved in drug metabolism is the gene encoding thymidylate synthase (TS).

14. The method of claim 1, wherein the cancer is EGFR—positive metastatic colorectal cancer.

15. The method of claim 1, wherein the genetic polymorphism comprises is the A870G cyclin D1 (CCND1) genetic polymorphism.

16. The method of claim 1, wherein the cancer is metastatic colon cancer and the polymorphism is selected from the group consisting of lle-GSTP1, Werner 1074, Werner 1367 and T-lnterleukin-8 (IL-8).

17. The method of claim 1, wherein the therapeutic regimen comprises the administration of CPT-11 or its biological equivalent.

18. The method of claim 1, wherein the cancer is metastatic colon cancer and the polymorphism is selected from the group consisting of EGFR, IL-8 and VEGF.

19. The method of claim 1, wherein the therapeutic regimen comprises plantinum-based chemotherapy and the polymorphism is in a gene selected from the group consisting of XPD gene, GSTP1 gene, TS gene and COX-2 promoter.

20. The method of claim 19, wherein the cancer is non-small cell lung cancer and the polymorphism is the —765 G to C genomic polymorphism in the promoter region of COX-2 gene.

21. The method of claim 1, wherein the genotype is high expression of a gene selected from the group consisting of Dipyrimidine dehydrogenase (DPD), VEGF, Survivin and EGFR and the cancer is rectal cancer.

22. The method of claim 21, wherein the cancer is colorectal cancer and the genotype is high mRNA expression of EGFR or ERCC1.

23. A method for determining if a resected rectal cancer patient is likely to experience tumor recurrence after chemoradiation treatment, comprising detecting the presence or absence of an Ala-9 Val single nucleotide polymorphism in the mitochondral targeting region of the manganese superoxide dismutase (MnSOD) gene or a Pro/Leu single nucleotide polymorphism at codon 197 near the C-terminus of the glutathione peroxidase-1 (GPx-1) protein, in a cell or sample isolated from said patient, wherein detecting the presence of homozygous Ala for MnSOD or homozygous Leu for GPx-1, from the patient sample predicts likelihood of tumor recurrence in said patient.

24. A method for determining if a colorectal cancer patient is likely to experience distant metastases, comprising detecting the presence or absence of a functional polymorphism of a gene involved in the angiogenic pathway or separately the VEGF pathway, wherein the presence of the functional polymorphism is predictive of the likelihood to experience distant metastases.

25. The method of claim 24, wherein the functional polymorphism is a +869 Leu/Pro TGF-β or -1607 G/2G MMP-1.

26. The method of claim 24, wherein the functional polymorphism is of a gene involved in the VEGF pathway.

27. The method of claim 24, wherein the gene is selected from the group consisting of EGFR, IL-8 and VEGF.

28. A method for predicting disease aggression survival time in female colorectal cancer patients determining under the age of 40, comprising the number of (CA) base pair repeats in the 3′ noncoding region in the estrogen receptor beta (Erβ) gene.


SEQUENCE LISTING (84 sequences)

SEQ ID NO: 1 - DNA, 22 bp, Artificial Sequence
  Description of Artificial Sequence Synthetic primer
gtgaagttcatttccaatccgc

SEQ ID NO: 2 - DNA, 20 bp, Artificial Sequence
  Description of Artificial Sequence Synthetic primer
ccgcttcctttgtccatcag

SEQ ID NO: 3 - DNA, 20 bp, Artificial Sequence
  Description of Artificial Sequence Synthetic primer
tgctgtgacccactctgtct

SEQ ID NO: 4 - DNA, 18 bp, Artificial Sequence
  Description of Artificial Sequence Synthetic primer
tgtcactaaaggaaagga

SEQ ID NO: 5 - DNA, 22 bp, Artificial Sequence
  Description of Artificial Sequence Synthetic primer
ttgttctaacacctgccactct

SEQ ID NO: 6 - DNA, 20 bp, Artificial Sequence
  Description of Artificial Sequence Synthetic primer
acaccatcaccatcgacaga

SEQ ID NO: 7 - DNA, 20 bp, Artificial Sequence
  Description of Artificial Sequence Synthetic primer
gggacatcaccctcacttac

SEQ ID NO: 8 - DNA, 23 bp, Artificial Sequence
  Description of Artificial Sequence Synthetic primer
ggctgtatatctgctctatatgc

SEQ ID NO: 9 - DNA, 20 bp, Artificial Sequence
  Description of Artificial Sequence Synthetic primer
ccagaaggttgcacttgtcc

SEQ ID NO: 10 - DNA, 20 bp, Artificial Sequence
  Description of Artificial Sequence Synthetic primer
ttcacagagtttaacagccc

SEQ ID NO: 11 - DNA, 20 bp, Artificial Sequence
  Description of Artificial Sequence Synthetic primer
ggcaaacctgagtcatcaca

SEQ ID NO: 12 - DNA, 20 bp, Artificial Sequence
  Description of Artificial Sequence Synthetic primer
tcggtgatttagcagcaaga

SEQ ID NO: 13 - DNA, 17 bp, Artificial Sequence
  Description of Artificial Sequence Synthetic primer
gagcgcggctacagctt

SEQ ID NO: 14 - DNA, 23 bp, Artificial Sequence
  Description of Artificial Sequence Synthetic primer
gctcaaacatgatgtttgcattc

SEQ ID NO: 15 - DNA, 20 bp, Artificial Sequence
  Description of Artificial Sequence Synthetic primer
tgcatgttcgtggcctctaa

SEQ ID NO: 16 - DNA, 18 bp, Artificial Sequence
  Description of Artificial Sequence Synthetic primer
tgcgtctcttgccggaat

SEQ ID NO: 17 - DNA, 23 bp, Artificial Sequence
  Description of Artificial Sequence Synthetic primer
cagctctgtgtgaaggtgcagtt

SEQ ID NO: 18 - DNA, 18 bp, Artificial Sequence
  Description of Artificial Sequence Synthetic primer
agtggtcccaggctgcac

SEQ ID NO: 19 - DNA, 17 bp, Artificial Sequence
  Description of Artificial Sequence Synthetic primer
gagcgcggctacagctt

SEQ ID NO: 20 - DNA, 23 bp, Artificial Sequence
  Description of Artificial Sequence Synthetic primer
gctcaaacatgatgtttgcattc

SEQ ID NO: 21 - DNA, 19 bp, Artificial Sequence
  Description of Artificial Sequence Synthetic primer
aggacgcaaggagggtttg

SEQ ID NO: 22 - DNA, 18 bp, Artificial Sequence
  Description of Artificial Sequence Synthetic primer
tgcgtctcttgccggaat

SEQ ID NO: 23 - DNA, 21 bp, Artificial Sequence
  Description of Artificial Sequence Synthetic primer
gggaatttggcgacgtaattc

SEQ ID NO: 24 - DNA, 24 bp, Artificial Sequence
  Description of Artificial Sequence Synthetic primer
cctgtaccagtccaataccatcct

SEQ ID NO: 25 - DNA, 23 bp, Artificial Sequence
  Description of Artificial Sequence Synthetic primer
cagctctgtgtgaaggtgcagtt

SEQ ID NO: 26 - DNA, 18 bp, Artificial Sequence
  Description of Artificial Sequence Synthetic primer
gtcccaggagcccatcct

SEQ ID NO: 27 - DNA, 20 bp, Artificial Sequence
  Description of Artificial Sequence Synthetic primer
tgtagcaaagatgccaaggt

SEQ ID NO: 28 - DNA, 18 bp, Artificial Sequence
  Description of Artificial Sequence Synthetic primer
gcctcggtgtgcctttca

SEQ ID NO: 29 - DNA, 18 bp, Artificial Sequence
  Description of Artificial Sequence Synthetic primer
agtggtcccaggctgcac

SEQ ID NO: 30 - DNA, 22 bp, Artificial Sequence
  Description of Artificial Sequence Synthetic primer
tccttaatgtcacgcacgattt

SEQ ID NO: 31 - DNA, 18 bp, Artificial Sequence
  Description of Artificial Sequence Synthetic primer
gctggccctcgcttatga

SEQ ID NO: 32 - DNA, 20 bp, Artificial Sequence
  Description of Artificial Sequence Synthetic primer
gtccgccgagtccttactga

SEQ ID NO: 33 - DNA, 20 bp, Artificial Sequence
  Description of Artificial Sequence Synthetic primer
ggctcaccctccagaagctt

SEQ ID NO: 34 - DNA, 18 bp, Artificial Sequence
  Description of Artificial Sequence Synthetic primer
gcggaggctgaggaacag

SEQ ID NO: 35 - DNA, 20 bp, Artificial Sequence
  Description of Artificial Sequence Synthetic primer
tcctgctggtccttcccata

SEQ ID NO: 36 - DNA, 24 bp, Artificial Sequence
  Description of Artificial Sequence Synthetic primer
gggtggaaaggtttggagtatgtc

SEQ ID NO: 37 - DNA, 19 bp, Artificial Sequence
  Description of Artificial Sequence Synthetic primer
acccggctgttgtctccat

SEQ ID NO: 38 - DNA, 20 bp, Artificial Sequence
  Description of Artificial Sequence Synthetic primer
tgttatcatgccggacttct

SEQ ID NO: 39 - DNA, 17 bp, Artificial Sequence
  Description of Artificial Sequence Synthetic primer
cccgtgatgtgcgcaat

SEQ ID NO: 40 - DNA, 22 bp, Artificial Sequence
  Description of Artificial Sequence Synthetic primer
tccatgaacttcaccacttcgt

SEQ ID NO: 41 - DNA, 18 bp, Artificial Sequence
  Description of Artificial Sequence Synthetic probe
accaccacggccgagcgg

SEQ ID NO: 42 - DNA, 24 bp, Artificial Sequence
  Description of Artificial Sequence Synthetic probe
tgcccagcacttcacgcatcagtt

SEQ ID NO: 43 - DNA, 29 bp, Artificial Sequence
  Description of Artificial Sequence Synthetic probe
cagtgcctacagtctcgagtctgccagtg

SEQ ID NO: 44 - DNA, 21 bp, Artificial Sequence
  Description of Artificial Sequence Synthetic probe
acgcattccctgcctcggctg

SEQ ID NO: 45 - DNA, 25 bp, Artificial Sequence
  Description of Artificial Sequence Synthetic probe
cacaggtgctctggcccagcacata

SEQ ID NO: 46 - DNA, 20 bp, Artificial Sequence
  Description of Artificial Sequence Synthetic probe
tcacctgggccgcacccttg

SEQ ID NO: 47 - DNA, 35 bp, Artificial Sequence
  Description of Artificial Sequence Synthetic probe
tgcactgacatctaagttctttagcactccttggc

SEQ ID NO: 48 - DNA, 27 bp, Artificial Sequence
  Description of Artificial Sequence Synthetic probe
actgcagcattgctgagaacattgcct

SEQ ID NO: 49 - DNA, 28 bp, Artificial Sequence
  Description of Artificial Sequence Synthetic probe
ccttctcctcctccaggacataagtgga

SEQ ID NO: 50 - DNA, 21 bp, Artificial Sequence
  Description of Artificial Sequence Synthetic probe
tcgccagctacgccctgctca

SEQ ID NO: 51 - DNA, 26 bp, Artificial Sequence
  Description of Artificial Sequence Synthetic probe
atggcagaaggaggagggcagaatca

SEQ ID NO: 52 - DNA, 22 bp, Artificial Sequence
  Description of Artificial Sequence Synthetic primer
tccttaatgtcacgcacgattt

SEQ ID NO: 53 - DNA, 18 bp, Artificial Sequence
  Description of Artificial Sequence Synthetic primer
gctggccctcgcttatga

SEQ ID NO: 54 - DNA, 22 bp, Artificial Sequence
  Description of Artificial Sequence Synthetic primer
tcggtgtagatgcacagcttct

SEQ ID NO: 55 - DNA, 20 bp, Artificial Sequence
  Description of Artificial Sequence Synthetic primer
ggctcaccctccagaagctt

SEQ ID NO: 56 - DNA, 24 bp, Artificial Sequence
  Description of Artificial Sequence Synthetic primer
gggtggaaaggtttggagtatgtc

SEQ ID NO: 57 - DNA, 22 bp, Artificial Sequence
  Description of Artificial Sequence Synthetic primer
tccatgaacttcaccacttcgt

SEQ ID NO: 58 - DNA, 18 bp, Artificial Sequence
  Description of Artificial Sequence Synthetic probe
accaccacggccgagcgg

SEQ ID NO: 59 - DNA, 24 bp, Artificial Sequence
  Description of Artificial Sequence Synthetic probe
tgcccagcacttcacgcatcagtt

SEQ ID NO: 60 - DNA, 23 bp, Artificial Sequence
  Description of Artificial Sequence Synthetic probe
aaggagaccatccccctgacggc

SEQ ID NO: 61 - DNA, 21 bp, Artificial Sequence
  Description of Artificial Sequence Synthetic probe
acgcattccctgcctcggctg

SEQ ID NO: 62 - DNA, 35 bp, Artificial Sequence; identical to SEQ ID NO: 47
  Description of Artificial Sequence Synthetic probe

SEQ ID NO: 63 - DNA, 26 bp, Artificial Sequence
  Description of Artificial Sequence Synthetic probe
atggcagaaggaggagggcagaatca

SEQ ID NO: 64 - DNA, 18 bp, Artificial Sequence
  Description of Artificial Sequence Synthetic primer
gcctcggtgtgcctttca

SEQ ID NO: 65 - DNA, 17 bp, Artificial Sequence
  Description of Artificial Sequence Synthetic primer
cccgtgatgtgcgcaat

SEQ ID NO: 66 - DNA, 21 bp, Artificial Sequence
  Description of Artificial Sequence Synthetic probe
tcgccagctacgccctgctca

SEQ ID NO: 67 - DNA, 22 bp, Artificial Sequence
  Description of Artificial Sequence Synthetic primer
tcactggcagactcgagactgt

SEQ ID NO: 68 - DNA, 18 bp, Artificial Sequence
  Description of Artificial Sequence Synthetic primer
tggccgaagtggaacaca

SEQ ID NO: 69 - DNA, 25 bp, Artificial Sequence
  Description of Artificial Sequence Synthetic probe
ccgccgagtccttactgagcacagg

SEQ ID NO: 70 - DNA, 21 bp, Artificial Sequence
  Description of Artificial Sequence Synthetic primer
gggaatttggcgacgtaattc

SEQ ID NO: 71 - DNA, 18 bp, Artificial Sequence
  Description of Artificial Sequence Synthetic primer
gcggaggctgaggaacag

SEQ ID NO: 72 - DNA, 25 bp, Artificial Sequence
  Description of Artificial Sequence Synthetic probe
cacaggtgctctggcccagcacata

SEQ ID NO: 73 - DNA, 22 bp, Artificial Sequence
  Description of Artificial Sequence Synthetic primer
aggtgaaggaaaggccatgtac

SEQ ID NO: 74 - DNA, 26 bp, Artificial Sequence
  Description of Artificial Sequence Synthetic primer
catatgctacattatccaggacatca

SEQ ID NO: 75 - DNA, 27 bp, Artificial Sequence
  Description of Artificial Sequence Synthetic probe
tgccagagagaccatacctctcagcca

SEQ ID NO: 76 - DNA, 18 bp, Artificial Sequence
  Description of Artificial Sequence Synthetic primer
agtggtcccaggctgcac

SEQ ID NO: 77 - DNA, 22 bp, Artificial Sequence
  Description of Artificial Sequence Synthetic primer
tccatgaacttcaccacttcgt

SEQ ID NO: 78 - DNA, 26 bp, Artificial Sequence
  Description of Artificial Sequence Synthetic probe
atggcagaaggaggagggcagaatca

SEQ ID NO: 79 - DNA, 18 bp, Artificial Sequence
  Description of Artificial Sequence Synthetic primer
tgcgtctcttgccggaat

SEQ ID NO: 80 - DNA, 20 bp, Artificial Sequence
  Description of Artificial Sequence Synthetic primer
ggctcaccctccagaagctt

SEQ ID NO: 81 - DNA, 21 bp, Artificial Sequence
  Description of Artificial Sequence Synthetic probe
acgcattccctgcctcggctg

SEQ ID NO: 82 - DNA, 18 bp, Artificial Sequence
  Description of Artificial Sequence Synthetic primer
tgagcgcggctacagctt

SEQ ID NO: 83 - DNA, 22 bp, Artificial Sequence
  Description of Artificial Sequence Synthetic primer
tccttaatgtcacgcacgattt

SEQ ID NO: 84 - DNA, 18 bp, Artificial Sequence
  Description of Artificial Sequence Synthetic probe
accaccacggccgagcgg